SEQUENCE LISTING (28 sequences)

SEQ ID NO: 1 - protein, 6 aa, synthetic construct
  REGION 1..6: note = Synthetic polypeptide
SDYWMS

SEQ ID NO: 2 - protein, 16 aa, synthetic construct
  REGION 1..16: note = Synthetic polypeptide
NIKYDGSEKYYVDSVK

SEQ ID NO: 3 - protein, 13 aa, synthetic construct
  REGION 1..13: note = Synthetic polypeptide
AGLLWFGEKAFDI

SEQ ID NO: 4 - protein, 5 aa, synthetic construct
  REGION 1..5: note = Synthetic polypeptide
SRYLA

SEQ ID NO: 5 - protein, 11 aa, synthetic construct
  REGION 1..11: note = Sythetic polypeptide
YDASNRATGIP

SEQ ID NO: 6 - protein, 9 aa, synthetic construct
  REGION 1..9: note = Synthetic polypeptide
QQRRSWPPT

SEQ ID NO: 7 - protein, 120 aa, synthetic construct
  REGION 1..120: note = Synthetic polypeptide
EVQLVESGGGLVQPGGSLRLSCAASGFTFSDYWMSWVRQAPGKGLEWVANIKYDGSEKYYVDSVKGRFTTSRDNAKNSLYLQMNSLRAEDTAVYYCAGLLWFGEKAFDIWGQGTMVTVSS

SEQ ID NO: 8 - protein, 107 aa, synthetic construct
  REGION 1..107: note = Synthetic polypeptide
EIVLTQSPATLSLSPGERATLSCRASQNVSRYLAWYQQKPGQAPRLLIYDASNRATGIPARFSGSGSGTDFTLTISSLEPEDFAVYYCQQRRSWPPTFGGGTKVEIK

SEQ ID NO: 9 - protein, 5 aa, synthetic construct
  REGION 1..5: note = Synthetic polypeptide
ADPNR

SEQ ID NO: 10 - protein, 8 aa, synthetic construct
  REGION 1..8: note = Synthetic polypeptide
GGTLRTYG

SEQ ID NO: 11 - protein, 8 aa, synthetic construct
  REGION 1..8: note = Synthetic polypeptide
TIPLFGKT

SEQ ID NO: 12 - protein, 15 aa, synthetic construct
  REGION 1..15: note = Synthetic polypeptide
ARDLTTLTSYNWWDL

SEQ ID NO: 13 - protein, 7 aa, synthetic construct
  REGION 1..7: note = Synthetic polypeptide
QSVTSSQ

SEQ ID NO: 14 - skipped

SEQ ID NO: 15 - protein, 8 aa, synthetic construct
  REGION 1..8: note = Synthetic polypeptide
QQYGSSPT

SEQ ID NO: 16 - protein, 121 aa, synthetic construct
  REGION 1..121: note = Synthetic polypeptide
QVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVS

SEQ ID NO: 17 - protein, 108 aa, synthetic construct [sequence omitted]

SEQ ID NO: 18 - protein, 394 aa, Simplexvirus .sp
MGGAAARLGAVILFVVIVGLHGVRGKYALVDASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPYHPPATPNNMGLIAGAVGGSLLAALVICGIVYWMRRHTQKAPKRIRLPHIREDDQPSSHQPLFY

SEQ ID NO: 19 - protein, 393 aa, Simplexvirus .sp
MGRLTSGVGTAALLVVAVGLRVVCAKYALADPSLKMADPNRFRGKNLPVLDRLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHHAPAAPSNPGLIIGALAGSTLAVLVIGGIAFWVRRRAQMAPKRLRLPHIRDDDAPPSHQPLFY

SEQ ID NO: 20 - protein, 5 aa, synthetic construct
SYAIN

SEQ ID NO: 21 - protein, 17 aa, synthetic construct
GLMPIFGTTNYAQKFQD

SEQ ID NO: 22 - protein, 16 aa, synthetic construct
VAYMLEPTVTAGGLDV

SEQ ID NO: 23 - protein, 12 aa, synthetic construct
RASQSVSSAYLA

SEQ ID NO: 24 - protein, 7 aa, synthetic construct
GASSRAT

SEQ ID NO: 25 - protein, 8 aa, synthetic construct
QQYGRSPT

SEQ ID NO: 26 - protein, 125 aa, synthetic construct
QVQLVQSGAEVKKPGSSVKVSCKASGGSFSSYAINWVRQAPGQGLEWMGGLMPIFGTTNYAQKFQDRLTITADVSTSTAYMQLSGLTYEDTAMYYCARVAYMLEPTVTAGGLDVWGQGTTVTVSS

SEQ ID NO: 27 - protein, 107 aa, synthetic construct
EIVLTQSPGTLSLSPGERATLSCRASQSVSSAYLAWYQQKPGQAPRLLIYGASSRATGIPDRFSGSGSGTDFTLTISRLEPEDFAVYYCQQYGRSPTFGGGTKVEIK

SEQ ID NO: 28 - protein, 225 aa, synthetic construct
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2025/0101084 A1
(43) Pub. Date: Mar. 27, 2025

(54) COMPOSITIONS AND METHODS FOR PREVENTING OR AMELIORATING NEONATAL HSV INFECTION

(71) Applicant: TRUSTEES OF DARTMOUTH COLLEGE, Hanover, NH (US)

(72) Inventors: Margaret E. ACKERMAN, Hanover, NH (US); Iara M. BACKES, Hanover, NH (US); David A. LEIB, Hanover, NH (US)

(21) Appl. No.: 18/725,123

(22) PCT Filed: Jan. 6, 2023
(86) PCT No.: PCT/US2023/060219

§ 371 Date: Jun. 27, 2024

Related U.S. Application Data
(60) Provisional application No. 63/377,063, filed on Sep. 26, 2022.
(60) Provisional application No. 63/297,417, filed on Jan. 7, 2022.

(51) Int. Cl. C07K 16/08; A61K 39/00; A61P 31/22

(52) U.S. Cl. CPC C07K 16/087; A61P 31/22; A61K 2039/505; A61K 2039/55; C07K 2317/24; C07K 2317/732

(57) ABSTRACT

The present disclosure relates to anti-HSV (herpes simplex virus) antibodies comprising an Fc region having at least one modification or mutation that confers enhanced effector function and/or improved binding to viral Fc receptor (vFcR) and uses of such antibodies for preventing or ameliorating the effects of a neonatal HSV infection.

### CROSS REFERENCE TO RELATED APPLICATIONS

This patent application claims priority to U.S. Provisional Patent Application No. 63/297,417, filed on Jan. 7, 2022 and U.S. Provisional Patent Application No. 63/377,063, filed on Sep. 26, 2022, the entire contents of which are fully incorporated herein by reference.

### FEDERALLY SPONSORED RESEARCH OR DEVELOPMENT

This invention was made with government support under R21 AI147714 awarded by the National Institutes of Health, R01 EY009083 awarded by the National Institutes of Health, and P01 AI098681 awarded by National Institutes of Health. The government has certain rights in the invention.

### SEQUENCE LISTING

The computer-readable Sequence Listing submitted on Jan. 6, 2023 and identified as follows: 32,377 bytes ST.26 XML document file named “029511-8105 Sequence Listing.xml,” created Jan. 6, 2023, is incorporated herein by reference in its entirety.

### TECHNICAL FIELD

The present invention relates to antibodies having enhanced effector function(s) and/or altered binding to the viral Fc receptor (vFcR) and the use of such antibodies for preventing, or ameliorating the neurological and/or non-neurological sequelae of, herpesvirus infection (e.g., herpes simplex virus (HSV) infection) and particularly neonatal herpesvirus infection (e.g., neonatal HSV infection).

### BACKGROUND

Neonatal herpes simplex virus (nHSV) infections are often devastating, resulting in significant mortality and morbidity despite antiviral therapy. Severe nHSV infections most often occur during or following birth in the absence of maternally transferred HSV-specific antibodies. The timing of therapeutic intervention is critical in the setting of nHSV, and delayed acyclovir treatment is associated with increased in-hospital death

Several monoclonal antibodies (mAbs) have shown to offer protection from acute HSV infection in adult preclinical animal models. Despite this evidence, however, little progress has been made towards Ab-based therapies to treat nHSV in the clinical setting.

Diverse Herpesviridae express IgG Fc region binding proteins. Glycoprotein E (gE) has pleiotropic activities, one of which is to function as a viral Fc receptor (vFcR), and is thought to assist in immune evasion from host Ab responses. The vFcR exists as a heterodimer with glycoprotein I (gE/gI) and is found on the viral envelope and the surface of infected cells. The gE/gI heterodimer forms a high affinity vFcR that binds monomeric IgG, while gE monomer binds IgG aggregates, forming a low affinity vFcR. Immune evasion mediated by the vFcR thought to take place through non-specific IgG shielding of viral epitopes and direct blocking of Ab effector functions. On the other hand, co-engagement of vFcR and FcγR may be possible. Thus, vFcR:Fc interactions are insufficiently understood, and better understanding is needed.

Indeed, HSV remains a significant danger to neonates, in part due to limitations of current antiviral treatment, lack of approved vaccines, and inconsistent implementation of newborn and/or birthing parent screening programs. Therefore, there is a great need to identify new therapeutic interventions for antenatal and perinatal infections, which have a tremendous potential to save lives or improve quality of life for many years.

### SUMMARY OF THE INVENTION

As disclosed herein, Fc-dependent mechanisms contribute to antibody-mediated protection against nHSV infection. For example, Fcγ receptor (FcγR) deficient mice are more susceptible to infection and decreased mAb Fc effector functions result in increased mortality. Data from both antibody Fc variants and FcγR deficient mice support the role of antibody effector function in the protective activities of diverse HSV-specific antibodies. Taken together, these results indicate that Fc-dependent mechanisms contribute to antibody-mediated protection against HSV infection.

Thus, in certain embodiments, the present disclosure contemplates Fc-modifications to anti-HSV antibodies to increase effector function(s). For example, an Fc modification can increase FcγRIIIa binding and/or C1q binding. As an example, the Fc region of a wild-type IgG1 back-bone can be modified to provide an afucosylated glycan at N297 as in HSV8-N reported herein. Afucosylated antibodies can be produced by, for example, cells in which a fucosyl transferase gene has been silenced or eliminated. For example, an afucosylated antibody may be produced in engineered tobacco plant cells (e.g., Nicotiana benthamiana). Without wishing to be bound by theory, it is believed that the afucosylated glycan at N297 increases binding to FcγRIIIa and increases ADCC. As another example, a mutation to the amino acid sequence of the Fc region of a wild-type IgG1 can increase binding to FcγRIIIa and/or C1q and provide increased effector function (e.g., increased ADCC, ADCP and/or CDC).

In one aspect, the present disclosure provides a method for protecting offspring against a neonatal viral infection, particularly infection by a vertically transmitted pathogen such as herpesvirus, and more particularly a HSV infection, such as an HSV-1 or HSV-2 infection and/or neurological or behavioral consequences thereof, the method comprising administering to a maternal subject that is pregnant or likely to become pregnant an anti-herpesvirus antibody, the anti-herpesvirus antibody comprising an Fc region having at least one modification or mutation that confers enhanced effector function.

In another aspect, the present disclosure provides a method for treating or preventing a neonatal viral infection, particularly infection by a vertically transmitted pathogen such as herpesvirus, and more particularly a HSV infection, such as an HSV-1 or HSV-2 infection and/or neurological or behavioral consequences thereof, the method comprising administering to a neonate infected with a herpesvirus, at risk for being infected with a herpesvirus, or that has been exposed to a herpesvirus an anti-herpesvirus antibody, the anti-herpesvirus antibody comprising an Fc region having at least one modification or mutation that confers enhanced effector function.

In certain embodiments of any aspect disclosed herein, the modification or mutation comprises an afucosylated Fc region.

In certain embodiments of any aspect disclosed herein, the modification or mutation comprises an amino acid mutation in the Fc region relative to wild-type IgG1 Fc region.

In certain embodiments of any aspect disclosed herein, the enhanced effector function comprises enhanced antibody dependent cellular cytotoxicity (ADCC).

In certain embodiments of any aspect disclosed herein, the enhanced effector function comprises enhanced antibody dependent cellular phagocytosis (ADCP).

In certain embodiments of any aspect disclosed herein, the enhanced effector function comprises enhanced complement-dependent cytotoxicity (CDC).

In certain embodiments of any aspect disclosed herein, the herpesvirus is HSV and the anti-herpesvirus antibody is an anti-HSV antibody.

In certain embodiments of any aspect disclosed herein, the anti-HSV antibody specifically binds to glycoprotein D (gD) of HSV. Exemplary amino acid sequences for gD are shown in SEQ ID NOs: 18-19. In some such embodiments, the anti-HSV antibody blocks the interaction between gD and herpesvirus entry mediator (HVEM). In some such embodiments, the anti-HSV antibody is a monoclonal antibody (mAb). For example, the anti-HSV antibody comprises the CDRs of mAb 5188 (also known as CH42), which binds gD residues that interface with the herpes virus entry mediator (HVEM) receptor (Wang et al. JVi, 2017). As another example the anti-HSV antibody comprises the CDRs of mAb E317 (also known as UB-621). As yet another example the anti-HSV antibody comprises the CDRs of mAb HSV8.

In certain embodiments of any aspect disclosed herein, the anti-HSV antibody is systemically administered to subject, such as by intravenous injection. Alternatively, or additionally, the anti-HSV antibody may be delivered to the maternal subject via vector-mediated delivery (e.g., a nucleic acid encoding the antibody is administered to the maternal subject). In some such embodiments, the vector-mediated delivery is AAV vector-mediated delivery.

In certain embodiments of any aspect disclosed herein, the maternal subject is HSV seronegative. In certain embodiments, the maternal subject is suspected of having a primary HSV infection.

In certain embodiments of any aspect disclosed herein, the anti-HSV antibody is administered to the maternal subject prior to parturition.

### BRIEF DESCRIPTION OF DRAWINGS

For a better understanding of the invention, reference may be made to embodiments shown in the following drawings. The components in the drawings are not necessarily to scale and related elements may be omitted, or in some instances proportions may have been exaggerated, so as to emphasize and clearly illustrate the novel features described herein. In addition, system components can be variously arranged, as known in the art.

FIG. 1A-1D. Fluorescently labeled mAb accumulates at the placental-fetal interface. To assess maternally administered Ab biodistribution, conjugated antibody was administered IV on day 15 or 16 of gestation, then 2-3 days later tissues were prepared for whole body imaging using the cryo-macrotome. (FIG. 1A) Background fluorescence levels in a pregnant dam not injected with conjugated Ab. (FIG. 1B) Accumulation of fluorescently labeled UB-621Ab in a pregnant dam 2 days following I.V. administration. (FIG. 1C) Accumulation of fluorescently labeled Ab in conceptuses that were harvested from the murine uterus, with maternal and fetal layers removed as indicated. (FIG. 1D) Control background fluorescence was determined in a pregnant dam not injected with conjugated Ab. Each dot represents the mean value of all voxels per slice segment for one animal, bar represents the geometric mean.

FIG. 2A-2B. Epitope map of HSV-specific mAbs that target the viral entry mediator gD. (FIG. 2A) Linear representation of the gD extracellular domain with the HVEM binding domain (yellow) and Nectin domains (black). UB-621 is derived from the original clone of E317. The exact contact residues of HSV8 are not known, but shown is an approximation based on reactivity against gD residues 235-275. (FIG. 2B) The gD ectodomain space filling structure (PDB: JMA1) with cell receptor binding domains and mAb epitopes denoted by specific colors shown in panel A.

FIG. 3A-3C. Prophylactic and therapeutic HSV-specific mAbs prevent nHSV-associated mortality. Antibodies were delivered IP to pregnant dams, or pups. Pups were challenged i.n. two days post-partum with indicated viral dose, then observed to DPI 21. (FIG. 3A) Survival of pups following CH42 or IgG control (ctrl) administration to pregnant dams five days before infection. (FIG. 3B) survival of pups following administration of E317 or IgG ctrl one day before infection in solid lines, or CH42 or IgG ctrl one day post infection in dashed lines. (FIG. 3C) Survival of pups following HSV8, UB-621, CH42, or IgG ctrl administration immediately before infection with HSV-1 or HSV-2. P values were corrected for multiple comparisons when more than one HSV-specific mAb was compared to IgG control using the Sidak method. * p<0.05, ** p<0.01, *** p<0.001. DPI, day post infection, PFU, plaque forming unit.

FIG. 4. Administration of CH42 reduces viral dissemination. Pregnant dams or pups received IP injections of CH42 or IgG ctrl antibody before or after viral challenge with a luciferase-expressing reporter HSV-1. Pups were imaged by IVIS on DPI 2, then imaged until DPI 8. Representative images follow the same two pups sequentially. (FIG. 4, top panel) Bioluminescence imaging of pups following CH42 or IgG ctrl administration to pregnant dams five days before infection. (FIG. 4, middle panel) Bioluminescence imaging of pups following IP mAb administration and immediate subsequent viral challenge. (FIG. 4, bottom panel) Bioluminescence imaging of pups following IP mAb administration one day after infection. Quantification of the virally-derived bioluminescence is shown on the right side in top, middle, and bottom panels. Statistical significance was determined by two-way ANOVA, and Šídák's method for multiple comparisons. ** p<0.01, *** p<0.001, **** p<0.0001

FIG. 5A-5B. Offspring of HSV-specific mAb treated dams are protected from behavioral morbidity. Pregnant B6 mice were administered CH42 or IgG ctrl mAbs, then progeny were challenged with luciferase expressing HSV-1, then behaviorally assessed at 5 weeks. (FIG. 5A) Anxiety-like behavior analysis via the OFT of adult mice infected on day two post-partum. Representative traces and heatmaps illustrate the pattern of movement, as well as the time spent in specific areas. (FIG. 5B) Thigmotaxis ratio of adult mice assessed for anxiety-like behavior via the OFT. Thigmotaxis is a measure of anxiety, a normal score=0.5, with higher scores indicating increased anxiety. * p<0.05

FIG. 6A-6D. VIP-derived HSV-specific transferred mAbs protect pups from nHSV mortality. VIPs encoding 4 different mAb sequences were administered to female mice. Progeny were assessed for antibody transfer and protection from viral challenge. (FIG. 6A) Schematic of AAV huIgG expression vector structure and experimental approach. (FIG. 6B) Detection of in vivo expressed huIgG in the serum of VIP-administered female mice from week 0 through 4. (FIG. 6C) Biodistribution of huIgG in the viscera, brain, trigeminal ganglia and serum of offspring of VIP-treated dams. Signal is reported as the fold increase in huIgG in treated pups relative to untreated controls. (FIG. 6D) Survival of progeny of VIP-administered dams challenged with 1×10⁴ PFU of HSV-1 two days post-partum. Statistical significance was determined by the Log-rank (Mantel-Cox) test, all HSV specific mAbs are compared to IgG control (IgG ctrl). P values were corrected for multiple comparisons when more than one HSV-specific mAb is compared to IgG control using Šídák's method. *** p<0.001

FIG. 7A-7D. Mice that lack FcγRs are more susceptible to infection. As shown in FIG. 7A-7D, antibodies were assessed for in vitro function and in vivo survival studies. FIG. 7A: mAbs were assessed for hFcyR III activation, a surrogate for ADCC using luciferase reporter Jurkat cells. FIG. 7B: mAbs were incubated at indicated dilutions for neutralization of HSV-1 luciferase reporter virus. FIG. 7C, 7D: For survival studies mAbs were delivered IP to pups (40 ug/pup), pups were challenged intranasally 2 days post-partum.

FIG. 8A-8B. Mice that lack FcγRs can overcome susceptibility with optimized neutralization of virus with neutralizing mAbs. Antibodies were incubated in complex with virus at indicated concentrations for one hour, then delivered intranasally to pups and assessed for in vivo survival studies. FIG. 8A: WT mice bearing FcγR receptors were challenged with 20 or 100 ug/pup as indicated. FIG. 8B: knockout mice lacking FcγR receptors are challenged with 20 or 100 ug/pup as indicated.

FIG. 9A-9D. Fc mutations impact binding to the vFcR but not to antigen. Antibodies were incubated with infected cells, or with antigens bound to plates or beads to carry out binding and effector assays. FIG. 9A: Three different Fc variants showing binding to antigen gD. FIG. 9B: Two different Fc variants showing binding to gE, the main component of the viral Fc Receptor. FIG. 9C: Fc variants assayed for neutralization potency using a bioluminescent HSV-1 virus. FIG. 9D: mAbs were assessed for hFcyR III (CD16) activation, a surrogate for ADCC using luciferase reporter Jurkat cells.

FIG. 10A-10D. Decreased mAb Fc effector functions result in increased mortality. For survival studies mAbs were delivered IP to pups (40 or 10 ug/pup), pups were challenged intranasally 2 days post-partum. FIG. 10A: Pups were administered 40 ug HSV8 variants IP, then immediately challenged with 1×10⁴ PFU of HSV-1. FIG. 10B: Pups were administered 10 ug HSV8 variants IP, then immediately challenged with 1×10⁴ PFU of HSV-1. FIG. 10C: Pups were administered 40 ug CH42 variants IP, then immediately challenged with 1×10⁴ PFU of HSV-1. FIG. 10D: Pups were administered 40 μg of mAbs IP, then immediately challenged with 3×10² PFU of HSV-2.

FIG. 11A-11B. Fc mutations that alter vFcR binding improve mAb neutralization. FIG. 11A: Plaque reduction neutralization test (PRNT) results observed following incubation with varying concentrations of HSV8 mAb variants. FIG. 11B: The concentration of mAb required to achieve 50% neutralization (EC50) of virus in vitro was improved (lower) for HSV Fc variants with mutations that increase binding to the vFcR.

FIG. 12A-12B. gE/gI mutant viruses are not differentially sensitive to Fc mutant mAbs in vitro and retain in vivo pathogenicity. FIG. 12A: The gE/gI mutated virus NSgE264 (an Ig binding knockout virus) was neutralized equivalently well by HSV8 LA and WT mAbs (left), whereas HSV8 LA exhibits enhanced neutralization of gE/gI intact virus (right). FIG. 12B: Survival following 1,000 PFU of gE/gI mutated virus NSgE264 challenge.

FIG. 13A-13B. Evaluation of HSV-1 gE binding to anti-HSV mAb at pH 7.4 and pH 6.2.

FIG. 14A-14B. Evaluation of human FcRn binding to anti-HSV mAb at pH 7.4 and pH 6.38.

FIG. 15A-15C. Evaluation of anti-HSV mAb neutralization potency. FIG. 15A: Plaque reduction neutralization test (PRNT) results observed following incubation of HSV-1 NS with varying concentrations of mAb variants. FIG. 15B: PRNT results observed following incubation of HSV-1 NS gE mutant with varying concentrations of mAb variants. FIG. 15C: PRNT results observed following incubation of HSV-1 NS gE Rescue with varying concentrations of mAb variants.

FIG. 16A-16C. Evaluation of mAb efficacy in vivo. For survival studies 10 ug of HSV8, HSV8 LA, or an isotype control (VRC01) were delivered intraperitoneally (IP) prior to intranasal challenge with 1×10⁴ PFU of HSV-1 NS (FIG. 16A), HSV-1 NS gE mutant (FIG. 16B), or HSV-1 NS gE Rescue (FIG. 16C). Pups were monitored for survival for 21 days.

### DETAILED DESCRIPTION

This detailed description is intended only to acquaint others skilled in the art with the present invention, its principles, and its practical application so that others skilled in the art may adapt and apply the invention in its numerous forms, as they may be best suited to the requirements of a particular use. This description and its specific examples are intended for purposes of illustration only. This invention, therefore, is not limited to the embodiments described in this patent application, and may be variously modified.

### A. Definitions

As used in the specification and the appended claims, unless specified to the contrary, the following terms have the meaning indicated:

The term “adjuvant” refers to agents or compounds that prolong, enhance, and/or accelerate an immune response.

The term “antibody” includes a glycoprotein comprising at least two heavy (H) chains and two light (L) chains inter-connected by disulfide bonds, or an immunologically effective fragment thereof. The term “immunologically effective (antibody) fragment” as used herein refers to a portion of an intact antibody comprising the antigen-binding site or variable region. While the portion does not necessarily include the constant heavy chain domains (i.e., CH2, CH3 or CH4, depending on the antibody isotype) of the Fc region of the intact antibody, preferred antibodies disclosed herein comprise an Fc region and, in particular, an Fc region having at least one modification or mutation that confers (a) enhanced effector function and/or (b) improved viral Fc receptor (vFcR) and/or glycoprotein E (gE) binding properties relative to a wild-type Fc region.

The term “gD” refers to HSV envelope glycoprotein encoded by US6 gene. The HSV gD glycoprotein is a multifunction protein with that helps to define viral host tropism. As used herein, the term “gD” includes isolated mature glycoprotein, peptide fragments thereof (e.g., truncated forms), and fusion protein formed with gD or a fragment thereof and another peptide. An exemplary gD protein is the HSV-1 gD protein, referred to herein as “gD1.” Another exemplary gD protein is the HSV-2 gD protein, referred to as “gD2.” As an example, a gD protein may have at least 90%, at least 95%, or at least 97%, or at least 98%, or at least 99%, or 100% identity with the sequence of SEQ ID NO: 18 and/or SEQ ID NO: 19.

The term “herpesvirus” refers to a group of viruses belonging to the family Herpesviridae and, in particular, those viruses in which humans are the primary host. Humans are the primary host for several herpesviruses, including herpes simplex virus 1 (HSV-1), herpes simplex virus 2 (HSV-2), varicella-zoster virus (VZV), Epstein-Barr virus (EBV), human cytomegalovirus (HCMV), human herpesvirus 6A and 6B (HHV-6A and HHV-6B), human herpesvirus-7 (HHV-7), and Kaposi's sarcoma herpes virus (KSHV). Throughout the application, “HSV” is used to collectively refer to HSV-1 and HSV-2.

The term “maternal subject” includes humans and other primates as well as other mammals. The term maternal subject includes, for example, a premenopausal female. In certain embodiments, the maternal subject is a human. In certain embodiments, the maternal subject is a human female of reproductive age. In some such embodiments, the maternal subject is HSV seronegative. In some such embodiments, the maternal subject is suspected of having a primary HSV infection.

The terms “treat”, “treating” and “treatment” refer to both therapeutic and preventative or prophylactic measures to alleviate or abrogate a condition, disorder, or disease and/or the attendant symptoms thereof.

In this application, the use of the disjunctive is intended to include the conjunctive. The use of definite or indefinite articles is not intended to indicate cardinality. In particular, a reference to “the” object or “a” and “an” object is intended to denote also one of a possible plurality of such objects. Further, the conjunction “or” may be used to convey features that are simultaneously present instead of mutually exclusive alternatives. In other words, the conjunction “or” should be understood to include “and/or”. The terms “includes,” “including,” and “include” are inclusive and have the same scope as “comprises,” “comprising,” and “comprise” respectively.

### B. Anti-Herpesvirus Antibodies and Uses Thereof

In one aspect, the present disclosure provides an anti-herpesvirus antibody, such as an anti-HSV antibody, and, in particular an anti-HSV antibody that comprises an Fc region having at least one modification or mutation that confers (a) enhanced effector function and/or (b) improved viral Fc receptor (vFcR) and/or glycoprotein E (gE) binding properties relative to a wild-type Fc region. Such anti-herpesvirus antibodies are useful to prevent or ameliorate the effects of a neonatal herpesvirus infection, such as a herpes simplex virus (HSV) infection.

The results exemplified herein demonstrate that five different mAbs to glycoprotein D (gD), whether maternally derived or through direct treatment, were protective to mouse pups. Both pre- and post-exposure mAb treatment significantly shortened infection and decreased viral loads.

Administration of CH42 to pregnant dams, whether recombinantly expressed or through VIP, was protective against lethal viral challenge, but afforded less protection when CH42 administration and lethal-dose viral challenge were performed simultaneously. UB-621 (aka E317) and afucosylated HSV8 were more protective than CH42 when administered directly to pups simultaneously with virus.

Fc mutations and modifications were made to assess the role of effector function(s) in the protective effects of anti-HSV mAbs. The Fc mutations and modifications examined herein include M428L/N434S (referred to as “LS”), which increases FcRn binding; M252Y/S254T/T256E (referred to as “YTE”), which increases FcRn binding and has also been noted to decrease effector function; L234A/L235A/P329G (referred to as “LALAPG”), which abrogates effector function; and an afucosylated Fc region, which increases binding to FcγRIIIa and increases ADCC.

LS, LA, and YTE also display some HSV-specific effects because HSV has a viral Fc receptor (vFcR) and these residues are in close proximity to the vFcR (gE)-Fc interface contact residues. Without wishing to be bound by theory, it is believed that for YTE, mutation of M252 to Y and T256 to E will introduce more bulky and longer side chains, respectively, which will impact H247 of vFcR (gE) and possibly also the main chain atoms of residues 243-245 in the case of the M252 to Y mutation and the main chain atoms of residues 339-341 in the case of the T256 to E mutation. Without wishing to be bound by theory, it is believed that for LS/LA, mutation of M428 to L introduces a less hydrophobic side chain leading to altered contacts at the interface and the N343 to S or A mutation will introduce a smaller side chain and impact the H-bond to the main chain atoms of A248 of the receptor. Data provided herein indicates that these mutations affect functional aspects of viral neutralization.

The results described herein indicate that interactions with the viral Fc receptor (gE/gI complex) as well as Ab-dependent effector mechanisms contribute to protection in neonates and could be enhanced through antibody engineering strategies, such as by Fc mutations and modifications. Such mutations and modifications may influence binding to vFcR and gE in particular, increase binding to FcγRIIIa and/or C1q, and provide increased effector function (e.g., increased ADCC, ADCP and/or CDC). One exemplary modification is an Fc region having an afucosylated glycan at Asn297. Exemplary amino acid mutation(s) include, but are not limited to, M428L/N434S (“LS”); M428L/N434A (“LA”); M252Y/S254T/T256E (“YTE”); S267E/H268F (“EF”); E333A; S298A/E333A/K334A (“AAA”); S239D/1332E; S239D/A330L/1332E; K326W/E333S; S267E/H268F/S324T (“EFT”); G236A/S267E/H268F/S324T/1332E (“EFTAE”); G236A/S239D/A330L/1332E (“GASDALIE”); E345K; E430G; T250Q/M428L (“QL”); P2571/Q311I (“II”); P2571/N434H (“IH”); and combinations thereof.

Thus, in certain embodiments for any of the aspects described herein, the anti-herpesvirus antibody comprises an Fc region having at least one modification or mutation. In some such embodiments, the modification or mutation is an amino acid mutation relative to a wild-type Fc region. In some such embodiments, the modification or mutation confers (a) enhanced effector function and/or (b) improved viral Fc receptor (vFcR) and/or glycoprotein E (gE) binding properties relative to a wild-type Fc region. In some such embodiments, the modification or mutation confers improved vFcR and/or gE binding properties relative to a wild-type Fc region.

In some such embodiments, the wild-type Fc region is a human IgG1 Fc region. In some such embodiments, the wild-type Fc region comprises the amino acid sequence of SEQ ID NO: 28, which represents position 223 to 447 of an antibody heavy chain polypeptide as identified by the EU numbering system according to Kabat.

SEQ ID NO: 28: THTCPPCP APELLGGPSV FLFPPKPKDT
LMISRTPEVT CVVVDVSHED PEVKFNWYVD GVEVHNAKTK
PREEQYNSTY RVVSVLTVLH QDWLNGKEYK CKVSNKALPA
PIEKTISKAK GQPREPQVYT LPPSRDELTK NQVSLTCLVK
GFYPSDIAVE WESNGOPENN YKTTPPVLDS DGSFFLYSKL
TVDKSRWQQG NVFSCSVMHE ALHNHYTQKS LSLSPGK.

In some such embodiments, the mutation is not AAA (S298A/E333A/K334A).

In certain embodiments of any aspect disclosed herein, the modification or mutation imparts modified vFcR- and/or gE-binding properties relative to the wild-type Fc region. Thus, in certain embodiments of any aspect disclosed herein, the anti-HSV antibody has modified vFcR- and/or gE-binding properties compared to the wild-type Fc region.

In certain embodiments of any aspect disclosed herein, the anti-herpesvirus antibody comprises an Fc region having the LS (i.e., M428L/N434S) mutation.

In certain embodiments of any aspect disclosed herein, the anti-herpesvirus antibody comprises an Fc region having the LA (i.e., M428L/N434A) mutation.

In certain embodiments of any aspect disclosed herein, the anti-herpesvirus antibody comprises an Fc region having the YTE (i.e., M252Y/S254T/T256E) mutation.

In certain embodiments of any aspect disclosed herein, the anti-herpesvirus antibody comprises an Fc region having the triple mutation S298A/E333A/K334A.

In certain embodiments of any aspect disclosed herein, the Fc region of an anti-herpesvirus antibody does not comprise the triple mutation known as AAA (S298A/E333A/K334A).

In certain embodiments of any aspect disclosed herein, the Fc region of the anti-herpesvirus antibody exhibits pH dependent binding to vFcR. For example, the affinity for binding to vFcR at physiological pH (i.e., pH 7.4) may be different than at endosomal pH (i.e., pH 6.0 or 5.5). In some such embodiments, the affinity for binding to vFcR at physiological pH (i.e., pH 7.4) may be enhanced relative to the affinity for binding to vFcR at endosomal pH (i.e., pH 6.0 or 5.5).

In certain embodiments for any of the aspects described herein, the anti-herpesvirus antibody is an anti-HSV antibody. In some such embodiments, the anti-HSV antibody specifically binds to an HSV protein or a fragment thereof. In some such embodiments, the anti-HSV antibody specifically binds to HSV gD or a fragment thereof. The anti-gD antibody may be a neutralizing antibody that, for example, blocks HSV binding to HVEM. Exemplary anti-gD antibodies include DL11, 1D3, 5157, 5158, 5159, 5160, 5188, 5190, 5192, E317, E425 and Y571, which are identified in, for example, Nicola, et al., J Virol, 72 (5): 3595-3601 (1998), US 2014/0302062 (Haynes), and U.S. Pat. No. 8,252,906 (Lai), each of which is herein incorporated by reference in its entirety.

In certain embodiments for any of the aspects described herein, the anti-HSV antibody comprises (i) an antibody having the heavy chain variable region and light chain variable region of mAb 5188, (ii) an antibody having the heavy chain CDRs (i.e., SEQ ID NOs: 1-3) and the light chain CDRs (i.e., SEQ ID NOs: 4-6) of mAb 5188, (iii) an antibody having the binding specificity of mAb 5188, (iv) an antibody having the heavy chain variable region and light chain variable region of mAb E317, (v) an antibody having the heavy chain CDRs (i.e., SEQ ID NOs: 10-12) and the light chain CDRs (i.e., SEQ ID NOs: 13-15) of mAb E317 (according to the IMGT nomenclature), (vi) an antibody having the binding specificity of mAb E317, (vii) an antibody having the heavy chain variable region and light chain variable region of mAb HSV8 (originally called AC8), (viii) an antibody having the heavy chain CDRs and the light chain CDRs of mAb HSV8, and/or (ix) an antibody having the binding specificity of mAb HSV8 and further comprises an Fc region having at least one modification or mutation that confers enhanced effector function.

According to US 2014/0302062 (Haynes), which is herein incorporated by reference in its entirety, mAb 5188 (CH42) comprises a heavy chain variable region having an amino acid sequence corresponding to H005188 (SEQ ID NO: 7) and a light chain variable region having an amino acid sequence corresponding to K003946 (SEQ ID NO: 8).

The anti-HSV antibody may specifically bind to an HSV protein, such as gD, a fragment thereof, or a variant thereof and comprise a variable heavy chain and/or variable light chain shown in Table 1. The anti-HSV antibody may specifically bind to an HSV protein, such as gD, a fragment thereof, or a variant thereof and comprise the heavy chain CDRs and/or light chain CDRs shown in Table 1.

TABLE 1
List of Amino Acid Sequences of VH and VL Regions
of Anti-gD Monoclonal Antibody (mAb) 5188 (CH42).

| | SEQ | |
|---|---|---|
| PROTEIN | ID | |
| REGION | NO. | SEQUENCE |
| H005188 (VH)- | 1 | SDYWMS |
| CDR1 | | |
| H005188 (VH)- | 2 | NIKYDGSEKYYVDSVK |
| CDR2 | | |
| H005188 (VH)- | 3 | AGLLWFGEKAFDI |
| CDR3 | | |
| K003946 (VL)- | 4 | SRYLA |
| CDR1 | | |
| K003946 (VL)- | 5 | YDASNRATGIP |
| CDR2 | | |
| K003946 (VL)- | 6 | QQRRSWPPT |
| CDR3 | | |
| H005188 (VH) | 7 | EVQLVESGGG LVQPGGSLRL SCAASGFTFS |
| | | DYWMSWVRQA PGKGLEWVAN IKYDGSEKYY |
| | | VDSVKGRFTT SRDNAKNSLY LOMNSLRAED |
| | | TAVYYCAGLL WFGEKAFDIW GQGTMVTVSS |
| K003946 (VL) | 8 | EIVLTQSPAT LSLSPGERAT LSCRASQNVS |
| | | RYLAWYQQKP GOAPRLLIYD ASNRATGIPA |
| | | RFSGSGSGTD FTLTISSLEP EDFAVYYCQQ |
| | | RRSWPPTFGG GTKVEIK |

In some such embodiments, the anti-HSV antibody binds to an epitope located at the N terminus of HSV-1 gD. In a particular embodiment, the anti-HSV antibody binds to an epitope located within amino acids 12 to 16 (ADPNR; SEQ ID NO: 9) of HSV-1 gD.

According to U.S. Pat. No. 8,252,906 (Lai) and Lee, et al., Acta Crystallogr D Biol Crystallogr. 69 (10): 1935-1945 (2013), each of which is herein incorporated by reference in its entirety, mAb E317 comprises a heavy chain variable region having an amino acid sequence corresponding to SEQ ID NO: 16 and a light chain variable region having an amino acid sequence corresponding to SEQ ID NO: 17.

The anti-HSV antibody may specifically bind to an HSV protein, such as gD, a fragment thereof, or a variant thereof and comprise a variable heavy chain and/or variable light chain shown in Table 2. The anti-HSV antibody may specifically bind to an HSV protein, such as gD, a fragment thereof, or a variant thereof and comprise the heavy chain CDRs and/or light chain CDRs shown in Table 2.

TABLE 2
List of Amino Acid Sequences of VH and VL Regions
of Anti-gD Monoclonal Antibody (mAb) E317.

| | SEQ | |
|---|---|---|
| PROTEIN | ID | |
| REGION | NO. | SEQUENCE |
| E317 (VH)- | 10 | GGTLRTYG |
| CDR1 | | |
| E317 (VH)- | 11 | TIPLFGKT |
| CDR2 | | |
| E317 (VH)- | 12 | ARDLTTLTSYNWWDL |
| CDR3 | | |
| E317 (VL)- | 13 | QSVTSSQ |
| CDR1 | | |
| E317 (VL)- | 14 | GAS |
| CDR2 | | |
| E317 (VL)- | 15 | QQYGSSPT |
| CDR3 | | |
| E317 (VH) | 16 | QVTLKQSGAE VKKPGSSVKV SCTASGGTLR |
| | | TYGVSWVRQA PGQGLEWLGR TIPLFGKTDY |
| | | AQKFQGRVTI TADKSMDTSF MELTSLTSED |
| | | TAVYYCARDL TTLTSYNWWD LWGQGTLVTV |
| | | S |
| E317 (VL) | 17 | EIVLTQSPGT LSLSPGERAT LSCRASQSVT |
| | | SSQLAWYQQK PGQAPRLLIS GASNRATGIP |
| | | DRFSGSGSGT DFTLTISRLE PEDFAVYYCQ |
| | | QYGSSPTFGG GTKVEIKR |

According to Burioni et al., PNAS, 91 (1): 355-359 (1994), which is herein incorporated by reference in its entirety, AC8 (aka HSV8) is a recombinant human mAb recognizing HSV.

The anti-HSV antibody may specifically bind to an HSV protein, such as gD, a fragment thereof, or a variant thereof and comprise a variable heavy chain and/or variable light chain shown in Table 3. The anti-HSV antibody may specifically bind to an HSV protein, such as gD, a fragment thereof, or a variant thereof and comprise the heavy chain CDRs and/or light chain CDRs shown in Table 3.

TABLE 3
List of Amino Acid Sequences of VH and VL Regions
of Anti-gD Monoclonal Antibody (mAb) HSV8.

| | SEQ | |
|---|---|---|
| PROTEIN | ID | |
| REGION | NO. | SEQUENCE |
| HSV8 (VH)- | 20 | SYAIN |
| CDR1 | | |
| HSV8 (VH)- | 21 | GLMPIFGTTNYAQKFQD |
| CDR2 | | |
| HSV8 (VH)- | 22 | VAYMLEPTVTAGGLDV |
| CDR3 | | |
| HSV8 (VL)- | 23 | RASQSVSSAYLA |
| CDR1 | | |
| HSV8 (VL)- | 24 | GASSRAT |
| CDR2 | | |
| HSV8 (VL)- | 25 | QQYGRSPT |
| CDR3 | | |
| HSV8 (VH) | 26 | QVQLVQSGAE VKKPGSSVKV SCKASGGSFS |
| | | SYAINWVRQA PGQGLEWMGG LMPIFGTTNY |
| | | AQKFQDRLTI TADVSTSTAY MQLSGLTYED |
| | | TAMYYCARVA YMLEPTVTAG GLDVWGQGTT |
| | | VTVSS |
| HSV8 (VL) | 27 | EIVLTQSPGT LSLSPGERAT LSCRASQSVS |
| | | SAYLAWYQQK PGQAPRLLIY GASSRATGIP |
| | | DRFSGSGSGT DFTLTISRLE PEDFAVYYCQ |
| | | QYGRSPTFGG GTKVEIK |

In certain embodiments for any of the aspects described herein, the anti-HSV antibody comprises (i) an antibody having the heavy chain variable region and light chain variable region of HSV8 (i.e., SEQ ID NOs: 26-27), (ii) an antibody having the heavy chain CDRs (i.e., SEQ ID NOs: 20-22) and the light chain CDRs (i.e., SEQ ID NOs: 23-25) of HSV8, and/or (iii) an antibody having the binding specificity of HSV8 and further comprises an Fc region having at least one modification or mutation that confers enhanced effector function.

In certain embodiments for any of the aspects described herein, the anti-HSV antibody comprises (i) an antibody having the heavy chain variable region and light chain variable region of HSV8 (i.e., SEQ ID NOs: 26-27), (ii) an antibody having the heavy chain CDRs (i.e., SEQ ID NOs: 20-22) and the light chain CDRs (i.e., SEQ ID NOs: 23-25) of HSV8, and/or (iii) an antibody having the binding specificity of HSV8 and further comprises an Fc region having an afucosylated glycan at Asn297 and/or an amino acid mutation selected from the group consisting of M428L/N434S (“LS”), M428L/N434A (“LA”), M252Y/S254T/T256E (“YTE”), L234A/L235A/P329G (referred to as “LALAPG”), and combinations thereof.

In certain embodiments for any of the aspects described herein, the anti-HSV antibody comprises (i) an antibody having the heavy chain variable region and light chain variable region of HSV8 (i.e., SEQ ID NOs: 26-27), (ii) an antibody having the heavy chain CDRs (i.e., SEQ ID NOs: 20-22) and the light chain CDRs (i.e., SEQ ID NOs: 23-25) of HSV8, and/or (iii) an antibody having the binding specificity of HSV8 and further comprises an Fc region having an afucosylated glycan at Asn297 and/or an amino acid mutation selected from the group consisting of M428L/N434S (“LS”), M428L/N434A (“LA”), M252Y/S254T/T256E (“YTE”), and combinations thereof.

In certain embodiments for any of the aspects described herein, the anti-HSV antibody comprises (i) an antibody having the heavy chain variable region and light chain variable region of HSV8 (i.e., SEQ ID NOs: 26-27), (ii) an antibody having the heavy chain CDRs (i.e., SEQ ID NOs: 20-22) and the light chain CDRs (i.e., SEQ ID NOs: 23-25) of HSV8, and/or (iii) an antibody having the binding specificity of HSV8 and further comprises an Fc region having an afucosylated glycan at Asn297 and/or an amino acid mutation selected from the group consisting of M428L/N434S (“LS”), M428L/N434A (“LA”), and combinations thereof.

Exemplary anti-HSV antibodies include but are not limited to HSV8N (i.e., an afucosylated version of HSV8), HSV8 LS (i.e., an antibody having the sequence of HSV8 with the M428L/N434S (“LS”) mutation), HSV8 LA (i.e., an antibody having the sequence of HSV8 with the M428L/N434A (“LA”) mutation), HSV8N LS (i.e., an afucosylated version of HSV8 with the M428L/N434S (“LS”) mutation), and HSV8N LA (i.e., an afucosylated version of HSV8 with the M428L/N434A (“LA”) mutation).

In certain embodiments for any of the aspects described herein, the anti-HSV antibody is a monoclonal antibody. In certain embodiments for any of the aspects described herein, the anti-HSV antibody is a chimeric antibody, a single chain antibody, an affinity matured antibody, an Fc-modified antibody, an engineered antibody, a human antibody, a humanized antibody, or a fully human antibody.

The term “CDR” is used herein to refer to the “complementarity determining region” within an antibody variable sequence. There are three CDRs in each of the variable regions of the heavy chain and the light chain, which are designated “CDR1”, “CDR2”, and “CDR3”, for each of the variable regions. The term “CDR set” as used herein refers to a group of three CDRs that occur in a single variable region that binds the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as “Kabat CDRs”. Chothia and coworkers (Chothia and Lesk, J. Mol. Biol., 196:901-917 (1987); and Chothia et al., Nature, 342:877-883 (1989)) found that certain sub-portions within Kabat CDRs adopt nearly identical peptide backbone conformations, despite having great diversity at the level of amino acid sequence. These sub-portions were designated as “L1”, “L2”, and “L3”, or “H1”, “H2”, and “H3”, where the “L” and the “H” designate the light chain and the heavy chain regions, respectively. These regions may be referred to as “Chothia CDRs”, which have boundaries that overlap with Kabat CDRs. Other boundaries defining CDRs overlapping with the Kabat CDRs have been described by Padlan, FASEB J., 9:133-139 (1995), and MacCallum, J. Mol. Biol., 262 (5): 732-745 (1996). Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat- or Chothia-defined CDRs.

In one aspect, this disclosure provides a nucleic acid molecule encoding an antibody, preferably a monoclonal antibody or a fragment thereof, described herein.

In certain embodiments, the nucleic acid molecule comprises a nucleotide sequence encoding an anti-HSV antibody that comprises (i) a VH chain comprising three CDRs and (ii) a VL chain comprising three CDRs, wherein (VH)-CDR1 has the amino acid sequence of SEQ ID NO: 1, (VH)-CDR2 has the amino acid sequence of SEQ ID NO: 2, (VH)-CDR3 has the amino acid sequence of SEQ ID NO: 3, (VL)-CDR1 has the amino acid sequence of SEQ ID NO: 4, (VL)-CDR2 has the amino acid sequence of SEQ ID NO: 5, (VL)-CDR3 has the amino acid sequence of SEQ ID NO: 6. In certain embodiments, the nucleic acid molecule is contained in a vector.

In certain embodiments, the nucleic acid molecule comprises a nucleotide sequence encoding an anti-HSV antibody that comprises (i) a VH chain comprising three CDRs and (ii) a VL chain comprising three CDRs, wherein (VH)-CDR1 has the amino acid sequence of SEQ ID NO: 10, (VH)-CDR2 has the amino acid sequence of SEQ ID NO: 11, (VH)-CDR3 has the amino acid sequence of SEQ ID NO: 12, (VL)-CDR1 has the amino acid sequence of SEQ ID NO: 13, (VL)-CDR2 has the amino acid sequence of SEQ ID NO: 14, (VL)-CDR3 has the amino acid sequence of SEQ ID NO: 15. In certain embodiments, the nucleic acid molecule is contained in a vector.

In certain embodiments, the nucleic acid molecule comprises a nucleotide sequence encoding an anti-HSV antibody that comprises (i) a VH chain comprising three CDRs and (ii) a VL chain comprising three CDRs, wherein (VH)-CDR1 has the amino acid sequence of SEQ ID NO: 20, (VH)-CDR2 has the amino acid sequence of SEQ ID NO: 21, (VH)-CDR3 has the amino acid sequence of SEQ ID NO: 22, (VL)-CDR1 has the amino acid sequence of SEQ ID NO: 23, (VL)-CDR2 has the amino acid sequence of SEQ ID NO: 24, (VL)-CDR3 has the amino acid sequence of SEQ ID NO: 25. In certain embodiments, the nucleic acid molecule is contained in a vector.

In certain embodiments, the nucleic acid molecule comprises a nucleotide sequence encoding an anti-HSV antibody that comprises an amino acid mutation in the Fc region relative to wild-type IgG1 Fc region. In some such embodiments, the wild-type IgG1 Fc region comprises the amino acid sequence of SEQ ID NO: 28. In some such embodiments, the amino acid mutation is selected from the group consisting of LS (i.e., M428L/N434S), LA (i.e., M428L/N434A), and YTE (i.e., M252Y/S254T/T256E). Thus, the nucleic acid molecule may comprise a nucleotide sequence encoding an anti-HSV antibody that comprises an Fc region having the LS (i.e., M428L/N434S) mutation; an Fc region having the LA (i.e., M428L/N434A) mutation; or an Fc region having the YTE (i.e., M252Y/S254T/T256E) mutation.

In one aspect, this disclosure provides compositions, preferably pharmaceutically acceptable compositions, comprising the anti-HSV antibody described herein.

In certain embodiments, the anti-HSV antibody (or a nucleic acid and/or vector encoding the anti-HSV antibody) is a component in a pharmaceutical composition. In certain embodiments, the pharmaceutical composition comprising the antibody is administered systemically. In certain embodiments, the pharmaceutical composition comprising the antibody is administered intravenously or intramuscularly.

In some such embodiments, the pharmaceutical composition also contains a pharmaceutically acceptable carrier. Such pharmaceutical compositions comprising antibodies described herein are for use in preventing and/or ameliorating the effects of a neonatal HSV infection. In a specific embodiment, a composition comprises a monoclonal anti-HSV antibody described herein. Alternatively, a composition may comprise one or more anti-HSV antibodies described herein (e.g., a polyclonal population of anti-HSV antibodies). In accordance with these embodiments, the composition may further comprise of a carrier, diluent or excipient.

In certain embodiments, an anti-HSV antibody described herein is incorporated into pharmaceutical compositions suitable for administration to a subject. Typically, the pharmaceutical composition comprises an antibody described herein (such as, for example, an Fc-modified version of E317 or an Fc-modified version or HSV8 or an Fc-modified version of CH42) and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody.

In certain embodiments for any of the aspects described herein, the anti-HSV antibody is administered with one or more additional agents, e.g., a therapeutic agent (for example, a small molecule or biologic), said additional agent being selected by the skilled artisan for its intended purpose. For example, in one embodiment, the anti-HSV antibody is administered with an antiviral agent, particularly an anti-HSV agent such as acyclovir. Thus, in a further embodiment, a pharmaceutical composition disclosed herein may comprise at least one additional therapeutic agent for treating or preventing a viral infection.

### C. Specific Embodiments

(A1) A method for preventing or ameliorating the effects of a neonatal herpes simplex virus (HSV) infection comprising: (a) administering to a maternal subject that is pregnant or likely to become pregnant an anti-HSV antibody or (b) administering to a neonate infected with a HSV, at risk for being infected with a HSV, or that has been exposed to a HSV an anti-HSV antibody, wherein the anti-HSV antibody comprises an Fc region having at least one modification or mutation that confers enhanced effector function.

(A2) The method of embodiment A1, wherein the anti-HSV antibody comprises an afucosylated Fc region.

(A3) The method of embodiment A1, wherein the modification or mutation comprises an amino acid mutation in the Fc region relative to wild-type IgG1 Fc region.

(A4) The method of embodiment A3, wherein the enhanced effector function comprises enhanced antibody dependent cellular cytotoxicity (ADCC), enhanced antibody dependent cellular phagocytosis (ADCP), and/or enhanced complement-dependent cytotoxicity (CDC).

(A5) The method of any one of embodiments A1-A4, wherein the anti-HSV antibody has the binding specificity of mAb 5188, E317, or HSV8.

(A6) The method of any one of embodiments A1-A4, wherein the anti-HSV antibody comprises
a heavy chain variable region (VH) having
   a V_{H} CDR1 having an amino acid sequence of SEQ ID NO: 1,
   a V_{H} CDR2 having an amino acid sequence of SEQ ID NO: 2, and
   a V_{H} CDR3 having an amino acid sequence of SEQ ID NO: 3; and
a light chain variable region (VL) having
   a V_{L} CDR1 having an amino acid sequence of SEQ ID NO: 4,
   a V_{L} CDR2 having an amino acid sequence of SEQ ID NO: 5, and
   a V_{L} CDR3 having an amino acid sequence of SEQ ID NO: 6.

(A7) The method of any one of embodiments A1-A4, wherein the anti-HSV antibody comprises a heavy chain variable region (VH) having an amino acid sequence of SEQ ID NO: 7 and a light chain variable region (VL) having an amino acid sequence of SEQ ID NO: 8.

(A8) The method of any one of embodiments A1-A4, wherein the anti-HSV antibody comprises
a heavy chain variable region (VH) having
   a V_{H} CDR1 having an amino acid sequence of SEQ ID NO: 10,
   a V_{H} CDR2 having an amino acid sequence of SEQ ID NO: 11, and
   a V_{H} CDR3 having an amino acid sequence of SEQ ID NO: 12; and
a light chain variable region (VL) having
   a V_{L} CDR1 having an amino acid sequence of SEQ ID NO: 13,
   a V_{L} CDR2 having an amino acid sequence of SEQ ID NO: 14, and
   a V_{L} CDR3 having an amino acid sequence of SEQ ID NO: 15.

(A9) The method of any one of embodiments A1-A4, wherein the anti-HSV antibody comprises a heavy chain variable region (VH) having an amino acid sequence of SEQ ID NO: 16 and a light chain variable region (VL) having an amino acid sequence of SEQ ID NO: 17.

(A10) The method of any one of embodiments A1-A4, wherein the anti-HSV antibody comprises the heavy chain CDRs and the light chain CDRs of HSV8.

(A11) The method of any one of embodiments A1-A4, wherein the anti-HSV antibody comprises the heavy chain variable region (VH) and light chain variable region (VL) of HSV8.

(A12) The method of any of the preceding embodiments, wherein the maternal subject is HSV seronegative.

(A13) The method of any of the preceding embodiments, wherein the maternal subject is suspected of having a primary HSV infection.

(A14) The method of any of the preceding embodiments, wherein the maternal subject is pregnant.

(A15) The method of any of the preceding embodiments, wherein the anti-HSV antibody is administered to the maternal subject prior to parturition.

(B1) A method for preventing or ameliorating the effects of a neonatal herpes simplex virus (HSV) infection comprising: (a) administering to a maternal subject that is pregnant or likely to become pregnant an anti-HSV antibody or (b) administering to a neonate infected with a HSV, at risk for being infected with a HSV, or that has been exposed to a HSV an anti-HSV antibody, wherein the anti-HSV antibody comprises an Fc region having at least one modification or mutation that modifies viral Fc receptor (vFcR) and/or glycoprotein E (gE) binding properties relative to a wild-type Fc region.

(B2) The method of embodiment B1, wherein the modification or mutation comprises an amino acid mutation in the Fc region relative to wild-type IgG1 Fc region.

(B3) The method of embodiment B1, wherein the anti-HSV antibody comprises an Fc region having the LS (i.e., M428L/N434S) mutation; an Fc region having the LA (i.e., M428L/N434A) mutation; or an Fc region having the YTE (i.e., M252Y/S254T/T256E) mutation.

(B4) The method of any one of embodiments B1-B3, wherein the mutation confers altered (e.g., improved) vFcR and/or gE binding properties relative to a wild-type Fc region.

(B5) The method of any one of embodiments B1-B4, wherein the anti-HSV antibody has the binding specificity of mAb 5188, E317, or HSV8.

(B6) The method of any one of embodiments B1-B4, wherein the anti-HSV antibody comprises
a heavy chain variable region (VH) having
   a V_{H} CDR1 having an amino acid sequence of SEQ ID NO: 1,
   a V_{H} CDR2 having an amino acid sequence of SEQ ID NO: 2, and
   a V_{H} CDR3 having an amino acid sequence of SEQ ID NO: 3; and
a light chain variable region (VL) having
   a V_{L} CDR1 having an amino acid sequence of SEQ ID NO: 4,
   a V_{L} CDR2 having an amino acid sequence of SEQ ID NO: 5, and
   a V_{L} CDR3 having an amino acid sequence of SEQ ID NO: 6.

(B7) The method of any one of embodiments B1-B4, wherein the anti-HSV antibody comprises a heavy chain variable region (VH) having an amino acid sequence of SEQ ID NO: 7 and a light chain variable region (VL) having an amino acid sequence of SEQ ID NO: 8.

(B8) The method of any one of embodiments B1-B4, wherein the anti-HSV antibody comprises
a heavy chain variable region (VH) having
   a V_{H} CDR1 having an amino acid sequence of SEQ ID NO: 10,
   a V_{H} CDR2 having an amino acid sequence of SEQ ID NO: 11, and
   a V_{H} CDR3 having an amino acid sequence of SEQ ID NO: 12; and
a light chain variable region (VL) having
   a V_{L} CDR1 having an amino acid sequence of SEQ ID NO: 13,
   a V_{L} CDR2 having an amino acid sequence of SEQ ID NO: 14, and
   a V_{L} CDR3 having an amino acid sequence of SEQ ID NO: 15.

(B9) The method of any one of embodiments B1-B4, wherein the anti-HSV antibody comprises a heavy chain variable region (VH) having an amino acid sequence of SEQ ID NO: 16 and a light chain variable region (VL) having an amino acid sequence of SEQ ID NO: 17.

(B10) The method of any one of embodiments B1-B4, wherein the anti-HSV antibody comprises the heavy chain CDRs and the light chain CDRs of HSV8.

(B11) The method of any one of embodiments B1-B4, wherein the anti-HSV antibody comprises the heavy chain variable region (VH) and light chain variable region (VL) of HSV8.

(B12) The method of any of the preceding embodiments, wherein the maternal subject is HSV seronegative.

(B13) The method of any of the preceding embodiments, wherein the maternal subject is suspected of having a primary HSV infection.

(B14) The method of any of the preceding embodiments, wherein the maternal subject is pregnant.

(B15) The method of any of the preceding embodiments, wherein the anti-HSV antibody is administered to the maternal subject prior to parturition.

(C1) A method for preventing or ameliorating the effects of a herpes simplex virus (HSV) infection comprising: (a) administering an anti-HSV antibody to a subject that is infected with a HSV, at risk for being infected with a HSV, or that has been exposed to an HSV, wherein the anti-HSV antibody comprises an Fc region having at least one modification or mutation that modifies viral Fc receptor (vFcR) and/or glycoprotein E (gE) binding properties relative to a wild-type Fc region.

(C2) The method of embodiment C1, wherein the modification or mutation comprises an amino acid mutation in the Fc region relative to wild-type IgG1 Fc region.

(C3) The method of embodiment C1, wherein the anti-HSV antibody comprises an Fc region having the LS (i.e., M428L/N434S) mutation; an Fc region having the LA (i.e., M428L/N434A) mutation; or an Fc region having the YTE (i.e., M252Y/S254T/T256E) mutation.

(C4) The method of any one of embodiments C1-C3, wherein the mutation confers altered (e.g., improved) vFcR and/or gE binding properties relative to a wild-type Fc region.

(C5) The method of any one of embodiments C1-C4, wherein the anti-HSV antibody has the binding specificity of mAb 5188, E317, or HSV8.

(C6) The method of any one of embodiments C1-C4, wherein the anti-HSV antibody comprises
a heavy chain variable region (VH) having
   a V_{H} CDR1 having an amino acid sequence of SEQ ID NO: 1,
   a V_{H} CDR2 having an amino acid sequence of SEQ ID NO: 2, and
   a V_{H} CDR3 having an amino acid sequence of SEQ ID NO: 3; and
a light chain variable region (VL) having
   a V_{L} CDR1 having an amino acid sequence of SEQ ID NO: 4,
   a V_{L} CDR2 having an amino acid sequence of SEQ ID NO: 5, and
   a V_{L} CDR3 having an amino acid sequence of SEQ ID NO: 6.

(C7) The method of any one of embodiments C1-C4, wherein the anti-HSV antibody comprises a heavy chain variable region (VH) having an amino acid sequence of SEQ ID NO: 7 and a light chain variable region (VL) having an amino acid sequence of SEQ ID NO: 8.

(C8) The method of any one of embodiments C1-C4, wherein the anti-HSV antibody comprises
a heavy chain variable region (VH) having
   a V_{H} CDR1 having an amino acid sequence of SEQ ID NO: 10,
   a V_{H} CDR2 having an amino acid sequence of SEQ ID NO: 11, and
   a V_{H} CDR3 having an amino acid sequence of SEQ ID NO: 12; and
a light chain variable region (VL) having
   a V_{L} CDR1 having an amino acid sequence of SEQ ID NO: 13,
   a V_{L} CDR2 having an amino acid sequence of SEQ ID NO: 14, and
   a V_{L} CDR3 having an amino acid sequence of SEQ ID NO: 15.

(C9) The method of any one of embodiments C1-C4, wherein the anti-HSV antibody comprises a heavy chain variable region (VH) having an amino acid sequence of SEQ ID NO: 16 and a light chain variable region (VL) having an amino acid sequence of SEQ ID NO: 17.

(C10) The method of any one of embodiments C1-C4, wherein the anti-HSV antibody comprises the heavy chain CDRs and the light chain CDRs of HSV8.

(C11) The method of any one of embodiments C1-C4, wherein the anti-HSV antibody comprises the heavy chain variable region (VH) and light chain variable region (VL) of HSV8.

(C12) The method of any of the preceding embodiments, wherein the subject is HSV seronegative.

(C13) The method of any of the preceding embodiments, wherein the subject is suspected of having a primary HSV infection.

(C14) The method of any of the preceding embodiments, wherein the subject is pregnant.

(C15) The method of any of the preceding embodiments, wherein the subject is a neonate.

It will be readily apparent to those skilled in the art that other suitable modifications and adaptations of the compositions and methods of the invention described herein may be made using suitable equivalents without departing from the scope of the invention or the embodiments disclosed herein.

The compounds, compositions, and methods described herein will be better understood by reference to the following examples, which are included as an illustration of and not a limitation upon the scope of the invention.

### D. Examples

### Example 1: Maternally Transferred Monoclonal Antibodies Protect Neonatal Mice from Herpes Simplex Virus-Induced Mortality and Morbidity

Neonatal herpes simplex virus (nHSV) infections often result in significant mortality and neurological morbidity despite antiviral drug therapy. Maternally-transferred HSV-specific antibodies reduce the risk of clinically-overt nHSV, but this observation has not been translationally applied. In this Example, the hypothesis that HSV-specific human monoclonal antibodies (mAbs) can prevent mortality and morbidity associated with nHSV was tested using a neonatal mouse model. Using whole-body cryo-imaging of pregnant dams it was determined that mAbs were transferred to the progeny and accumulated at the maternal-fetal interface. Whether expressed by maternal AAV transduction or injected directly into dams, mAbs were efficiently transferred to pups and distributed to the nervous system. Through these maternally-derived routes or through direct administration to pups mAbs to HSV glycoprotein D protected against nHSV. Both pre- and post-exposure mAb treatment significantly reduced viral load as assessed by in vivo bioluminescent imaging. Administration of mAb also reduced nHSV-induced behavioral morbidity, as measured by anxiety-like behavior. Together these studies support the notion that HSV-specific mAb-based therapies may improve outcomes in neonates infected with HSV.

### Methods

Mouse procedures. C57BL/6 (B6) and B cell insufficient muMT (B6.129S2-Ighm^{tm1Cgn}/J) mice were purchased from The Jackson Laboratory. muMT mice were used in a subset of experiments to attribute protection to administered mAb, but results were interchangeable with the B6 mice which were therefore used for follow-up experiments. Blood collection was via cheek bleed from the mandibular vein with a 5 mm lancet for weanlings and adults, or a 25 G needle for 1-2 wk old pups. Animals <1 wk of age were euthanized prior to decapitation for blood collection. Blood samples were allowed to clot by stasis for ≥15 min. and then spun at 2000×g for 10 min. at 4° C. and supernatants collected and stored at −20° C. mAbs were administered intraperitoneally (i.p) to pups in 20 μl. mAb were administered i.p. to pregnant dams in volumes between 0.350-1 mL. For imaging studies, pups were injected i.p with 20 μl of 15 mg/ml D-luciferin potassium salt, placed in isoflurane chamber, and moved into the IVIS Xenogen with a warmed stage and continuous isoflurane. Pups were typically imaged 2 days post-infection and serially imaged every other day to monitor bioluminescence. Endpoints for survival studies were defined as excessive morbidity (hunched, spasms, or paralysis) or >10% weight loss.

Monoclonal antibodies. CH42 and CH43 plasmids were kindly provided by Dr. Tony Moody (Duke University). When expressed in vitro, CH42 contained the Fc mutation known as AAA (S298A/E333A/K334A), which enhances ADCC. See Shields R L et al. High Resolution Mapping of the Binding Site on Human IgG1 for FcγRI, FcγRII, FcγRIII, and FcRn and Design of IgG1 Variants with Improved Binding to the FcγR *. Journal of Biological Chemistry 2001; 276 (9): 6591-6604. E317 is the original clone of the clinical drug product UB-621; its heavy and light chain variable sequences were derived from published amino acid sequences (see WO2010087813A1) and synthesized in house as IDT gBlock for cloning onto IgG₁ backbones. In-house expressed antibodies were made through co-transfection of heavy and light chain plasmids in Expi293 HEK cells (Thermo Fisher) according to the manufacturer's instructions. Seven days after transfection, cultures were spun at 3000×g for 30 minutes to pellet the cells, and supernatants were filtered (0.22 μm). IgG was affinity purified using a custom packed 5 mL protein A column with a retention time of 1 minute (ie. 5 mL/min) and eluted with 100 mM glycine pH 3, which was immediately neutralized with 1 M Tris buffer pH 8. Eluate was then concentrated to 2.5 mL for size exclusion chromatography on a HiPrep Sephacryl S-200 HR column using an AktaPure FPLC at a flow rate of 1 mL/min of sterile PBS. Fractions containing monomeric IgG were pooled and concentrated using spin columns (Amicon UFC903024) to approximately 2 mg/mL of protein and either used within a week or aliquoted and frozen at −80° C. for later use. HSV8 mAb was kindly provided by ZabBio, this mAb has an IgG1 backbone and is Afucosylated. UB-621, a clinical grade antibody preparation with the E317 gene sequence expressed in hamster ovary cells, was kindly provided by United Biopharma.

Viral challenge. The wild-type viral strains used in this study were HSV-1 17syn+, HSV-2 G (kindly provided by Dr. David Knipe). The bioluminescent luciferase-expressing recombinant virus HSV-1 17syn+/Dlux was constructed as previously described. Viral stocks were prepared using Vero cells as previously described. Newborn pups were infected intranasally on day 1 or 2 postpartum with indicated amounts of HSV in a volume of 5 μl under isoflurane anesthesia. Pups were then monitored for survival, imaging, or behavior studies once adulthood was reached as appropriate. For survival studies, pups were challenged with 1×10³ or 1×10⁴ pfu of HSV-1 (Strain 17), and 3×10² HSV-2 (Strain G) as indicated. For imaging studies, pups were challenged with 1×10⁵ HSV-1 17syn+/Dlux.

Whole body cryo-macrotome imaging procedures. Conjugation of mAb UB-621 was as previously described. Briefly, 5 mg of mAb in 100 μL of PBS was incubated with 10 μl of filter-sterilized 1M sodium bicarbonate and 1 μl of 10 mg/mL AF488 NHS-ester (Lumiprobe) for 1 hr at room temperature and protected from light. Buffer exchange was carried out with Zeba spin columns (Thermo). Conjugation was confirmed through flow cytometry and spectrophotometer readings before animal experiments were performed. B6 dams were bred for timed pregnancies, and on day 11 of gestation chlorophyll-free diet (MP Biomedical) was initiated to reduce autofluorescence. On day 16 of gestation 5 mg AF488 labeled UB-621 was administered via tail-vein, and 2 days later animals were sacrificed and prepared for cryo-imaging by OCT (Tissue-Tek) flooding and subsequent freezing at −20° C. The hyperspectral imaging whole body cryo-macrotome instrument has been described previously. Briefly, the system operates by automatically sectioning frozen specimens in a slice-and-image sequence, acquiring images of the specimen block after each section is removed. For this study, we acquired brightfield and AF488 fluorescence volumes of each animal at a resolution of 150 μm in the sectioning direction and ˜100 μm in the imaging plane. Hyperspectral fluorescence images were spectrally unmixed using known fluorophore and tissue spectral bases to isolate the AF488 signal in animal tissue. The acquired image stacks were then combined in an open-source software platform (Slicer 4.11) to generate high-resolution three-dimensional volumes of the brightfield and fluorophore distribution throughout whole-body animal models.

Adeno-associated virus (AAV) production and procedure. AAVs encoding the heavy and light chain sequences of CH42, CH43, and E317, and control IgG mAbs were produced as previously described. See Balazs A B et al. Antibody-based protection against HIV infection by vectored immunoprophylaxis. Nature 2012; 481 (7379): 81-84. All AAV-derived mAbs were cloned with the same human IgG₁ backbone. A single 40 μl injection of 1×10¹¹ genome copies of AAV was administered into the gastrocnemius muscle of B6 or muMT mice as previously described. Blood samples were obtained by cheek bleed to verify antibody expression.

Assessment of mAb expression and biodistribution. A magnetic bead-based multiplex assay was used to measure antibody expression and biodistribution. Beads were conjugated to antigen or anti-human antigen-binding fragment (Fab) to capture mAbs of interest. Briefly, HSV gD (gD-2 (306) gifts from Gary Cohen, and Roselyn Eisenberg), HIV-1 gp140, or anti-human IgG F(ab′)2 fragment (Jackson Immune Research) were conjugated to fluorescent microspheres (MagPlex-C Microspheres, Luminex Corp.) at a ratio of 6.5 ug protein/100 μL microspheres. Samples were incubated with microspheres (500-750 beads/well) overnight at 4° C. and washed in PBS with 1% BSA, 0.05% Tween-20, and 0.1% sodium azide. Anti-human IgG PE (Southern Biotech) was incubated at 0.65 ug/ml for 45 min in PBS-TBN. The microspheres were washed and resuspended in 90 μl of sheath fluid (Luminex) and read using a Bio-plex array reader (FlexMap 3D, OR MAGPIX). The median fluorescence intensity (MFI) of the PE signal was determined for each sample at indicated dilutions. For biodistribution assessment signal is reported as the fold increase in PE signal in treated pups relative to untreated controls.

Behavioral tests and analysis. Animals were transferred to a dedicated behavior testing room at least one week before tests began. Environmental conditions, such as lighting, temperature, and noise levels were kept consistent. Behavioral tests and analysis were performed by independent, masked operators. The movement of animals was recorded (Canon Vixia HFM52) and videos were analyzed using open-source software. The Open Field Test was performed as previously described in Patel C D et al. Maternal immunization confers protection against neonatal herpes simplex mortality and behavioral morbidity. Sci. Transl. Med. 2019; 11 (487): eaau6039. Briefly, 5- to 7-week-old B6 mice were placed in the open field arena (30 cm×30 cm) and allowed to habituate for 10 mins before recording took place for an additional 10 mins.

Statistical Analysis. Prism 8 GraphPad software was used for statistical tests unless otherwise described. For survival studies, HSV-specific mAbs were compared to isotype controls using the Log-rank Mantel-Cox test to determine p values, if multiple comparisons took place, p values were corrected with the Holm-Sidak method. For imaging studies, groups and time points were compared to each other via two-way ANOVA, with Sidak's test for multiple comparisons to determine p values.

### Results

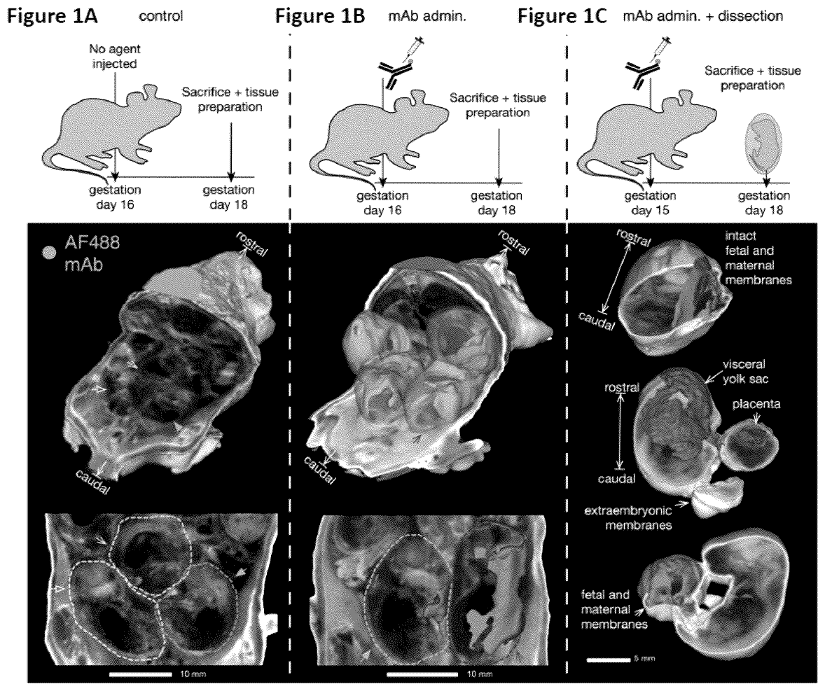

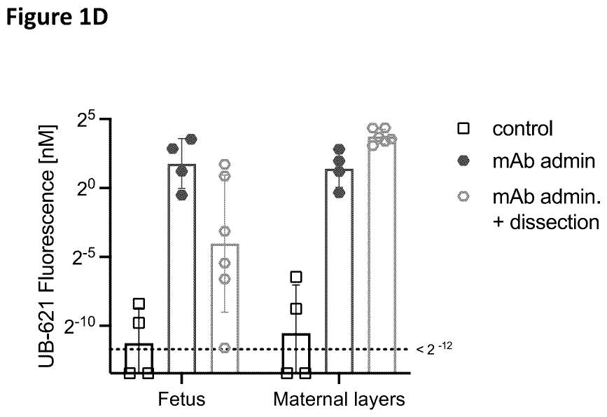

mAb UB-621 accumulates at the placental-fetal interface. While maternal Abs prevent nHSV mortality and morbidity, their biodistribution in pregnant dams has not been fully elucidated. To preserve the complex anatomy of the placental-fetal interface we pursued hyperspectral imaging via whole body cryo-macrotome processing, which causes minimal disruption to these tissues (FIG. 1A-1C). We administered fluorescently labeled UB-621 mAb to pregnant C57BL6 (B6) dams two days before sacrifice and tissue preparation. Two dams (naive and UB-621 infused) were immediately processed for cryo-imaging after sacrifice, while an additional dam was further dissected to prepare conceptuses for cryo-imaging. Robust fluorescent signal on tissues of the placental-fetal interface was detected in the cryo-imaged dam infused with fluorescent UB-621 as compared to the control dam that did not receive mAb (FIGS. 1A and 1B). To confirm that this signal originated from tissues at the placental-fetal interface, conceptuses were individually harvested from the mouse uterus and individually imaged with different layers of placental-fetal and/or fetal tissues dissected (FIG. 1C). These dissection experiments confirmed that mAb accumulated at the placental-fetal interface, and was also detected in fetal and maternal tissues (FIG. 1D). Notably strong fluorescence was observed in a harvested conceptus where the visceral yolk sac, a tissue rich in neonatal Fc receptor (FcRn) and necessary for IgG transfer, remained intact (FIG. 1C, middle image). Overall, this technique allowed us to visualize IgG traversing the circulation of the pregnant dam and entering maternal and fetal membranes, consistent with pronounced antibody accumulation at the maternal-fetal interface.

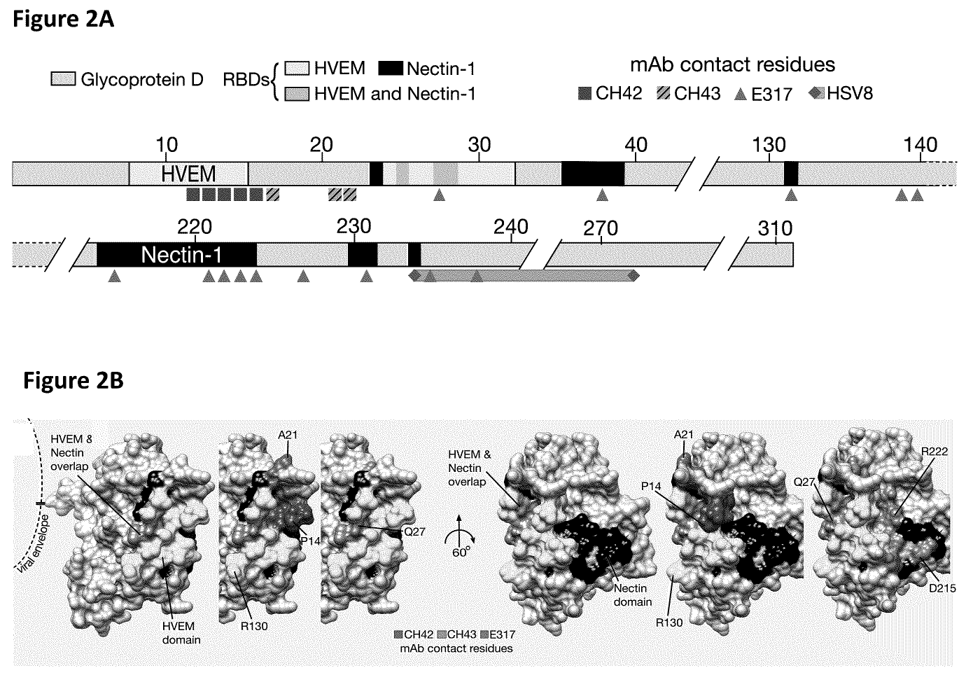

HSV mAbs targeting glycoprotein D protect neonatal mice from HSV-1 and HSV-2 mortality. The mAbs used in this study span the gD ectodomain, with epitopes close to the herpes virus entry mediator (HVEM) binding domain, and the Nectin (1 & 2) binding domains (FIG. 2A). The gD: mAb interfaces between E317/UB-621, CH42 and CH43 have been resolved in detail through crystallography and alanine scanning, while that of HSV8 is more broadly defined from binding experiments with truncated gD (FIG. 2B). All of these mAbs protect from HSV infection in adult mouse models (see Table 4).

TABLE 4
Brief summary of HSV-specific mAbs used in this study.

| mAb | Findings and summaries | Sources |
|---|---|---|
| CH42 | Reduced mortality & the establishment of latency | Wang, J Virol., 2017 |
| CH43 | in corneal HSV-1 challenge of adult mice. | US20140302062 |
| E317 | Reduced mortality in IP HSV-1 challenge of adult | Lee, Acta. Cryst. D, 2013 |
| | mice. | WO2010087813A1 |
| | | U.S. Pat. No. 8,431,118B2 |
| HSV8 | Reduced mortality in IP HSV-1 challenge of adult | Sanna, Virology, 1996 |
| | mice. Vaginal antibody safety trial: Safety study | Politch, PLoS med., 2021 |
| | of monoclonal antibodies to reduce the vaginal | NCT02579083 |
| | transmission of herpes simplex virus and human | |
| | immunodeficiency virus. Phase I completed. | |
| UB-621* | Phase 1 clinical trial for SC administration | NCT02346760 |
| | proving safe and well tolerated in healthy | NCT03595995 |
| | volunteers. | NCT04714060 |
| | Phase 2 clinical trials for the treatment of | NCT04979975 |
| | recurring genital HSV approved in the US and | Clementi, Drug Discov., 201 |
| | China. | |

*expressed in Chinese hamster ovarian cells from original clone E317

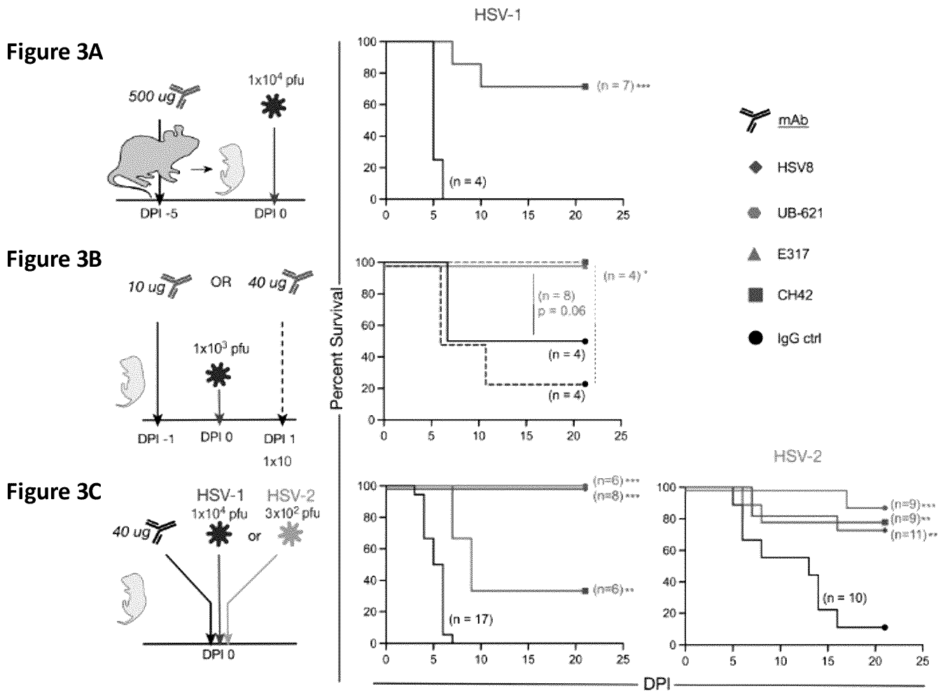

Like human neonates, mouse pups are highly susceptible to HSV infection, succumbing to infection at low viral doses relative to adult mice. Therefore, we wished to determine if HSV gD-specific mAbs could protect mouse pups from HSV-1 infection. Pregnant dams were administered either CH42 or control IgG approximately 3-5 days before parturition, and pups were challenged intranasally with HSV-1 one day after birth (FIG. 3A). Offspring of dams treated with CH42 showed significantly improved survival (p<0.001) compared to offspring of control IgG-treated dams.

While prophylactic approaches for nHSV are desirable we also sought to model therapeutic approaches to treat extant infections in neonates which may more closely model the clinical setting. To understand the prophylactic and therapeutic effect of mAb treatment, we administered E317 or control mAb one day before viral challenge, and given our prior results with prophylactic maternal CH42 treatment, CH42 was administered one day after viral challenge. Pups treated with E317 or CH42 exhibited improved survival (p=0.06 and p<0.05, respectively) relative to pups that received control IgG (FIG. 3B). We next assessed the protection afforded by mAbs currently being evaluated in clinical trials for adult genital HSV disease (HSV8 and UB-621). HSV8, UB-621, CH42, or control IgG were administered to pups, and immediately challenged with HSV-1. Both HSV8 and UB-621 mAbs completely protected pups from mortality following HSV-1 viral challenge (p<0.001), while CH42 afforded partial protection compared to control IgG-treated pups (p<0.01) (FIG. 3C, left panel). While HSV-1 genital disease predominates in the Americas and Western Pacific, and continues to rise as the etiologic agent of genital disease in high income countries, HSV-2 remains a significant cause of neonatal disease. Therefore, pups were treated with HSV8, UB-621 and CH42 as described above and challenged with HSV-2 (FIG. 3C, right panel). All HSV-specific mAbs tested resulted in significantly improved survival (UB-621 p<0.001, CH42 and HSV8 p<0.01) compared to control IgG-treated pups after viral challenge with HSV-2. Collectively, these data showed that gD specific mAbs can protect highly susceptible neonatal mice following administration with disparate routes, timings and doses of antibody, and following challenge with HSV-1 and HSV-2.

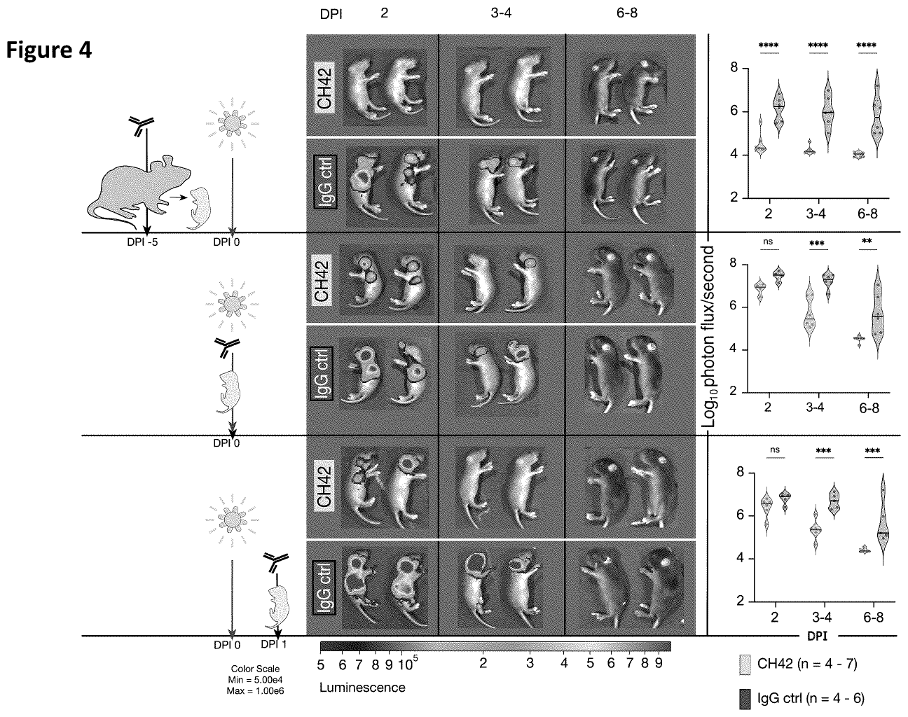

mAb CH42 reduces CNS and disseminated viral replication. Disseminated disease results in the highest case fatality rate among nHSV clinical presentations, and despite aggressive antiviral treatment, has an unacceptably high mortality (30%). We therefore assessed the impact of mAb therapy in the control of viral dissemination using bioluminescent imaging (BLI) to monitor viral replication and spread in real time (FIG. 4). Dams or pups received CH42 mAb or control IgG either before or after challenge with an HSV-1 recombinant that expressed luciferase. We performed BLI daily from 2 to 8 days post infection. As expected, virus was detected primarily in lungs, trigeminal ganglia, and brain consistent with BLI signals observed from intact mice (FIG. 4). Among the different treatment and timing modalities tested, offspring of mAb-treated dams cleared the infections fastest compared to control IgG (p<0.001), with nearly undetectable bioluminescence even at the earliest timepoint assessed (FIG. 4, top panel). When antibody and virus were administered simultaneously, or when antibody was administered one day following infection, a similar pattern was apparent in which bioluminesence was diminished in CH42-treated pups over time and reached background levels by day 6 (FIG. 4, middle and bottom panels). While mAb prophylaxis of the pregnant dam resulted in the most significant protection of pups, co- and post-infection mAb treatment also conferred protection demonstrating the efficacy of mAb therapy for nHSV.

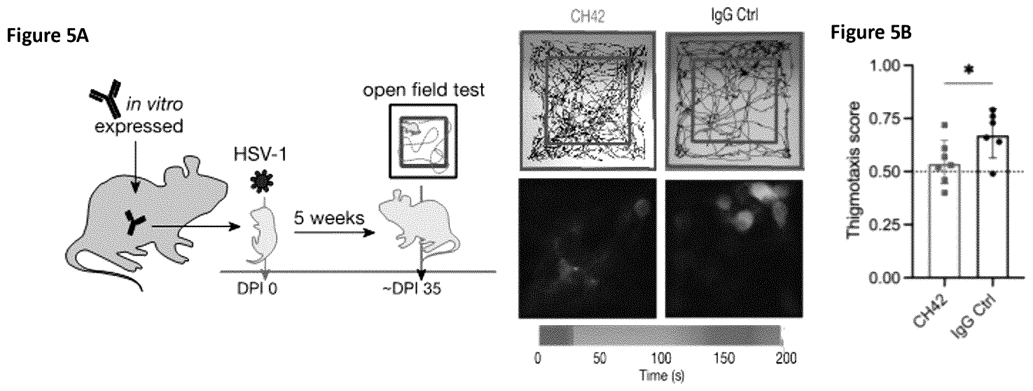

mAb immunotherapy reduces neurological morbidity in adult mice infected at birth. Neurological morbidity subsequent to HSV-1 infection of neonates was modeled using the Open Field Test (OFT, FIG. 5A), which analyzes the innate exploratory behavior of mice and measures anxiety-like behavior. Mice are placed in an enclosed arena and the time spent in the periphery relative to the total exploration time is measured (thigmotaxis ratio). Mice with anxiety-like behavior therefore have higher thigmotaxis ratios. Whereas scores of 0.5 are normal in B6 mice, HSV-infected neonates exhibit elevated thigmotaxis in adulthood. We therefore tested whether HSV-specific mAbs could protect mice from anxiety-like behavior that follows neonatal infection. Offspring of control IgG-treated dams spent significantly (p<0.05) more time in the periphery of the arena relative to progeny of CH42-treated dams (FIG. 5B). Offspring of CH42-treated dams showed thigmotaxis scores close to 0.5 (FIG. 5B), indicating that CH42 was able to protect mice from nHSV-induced behavioral morbidity.

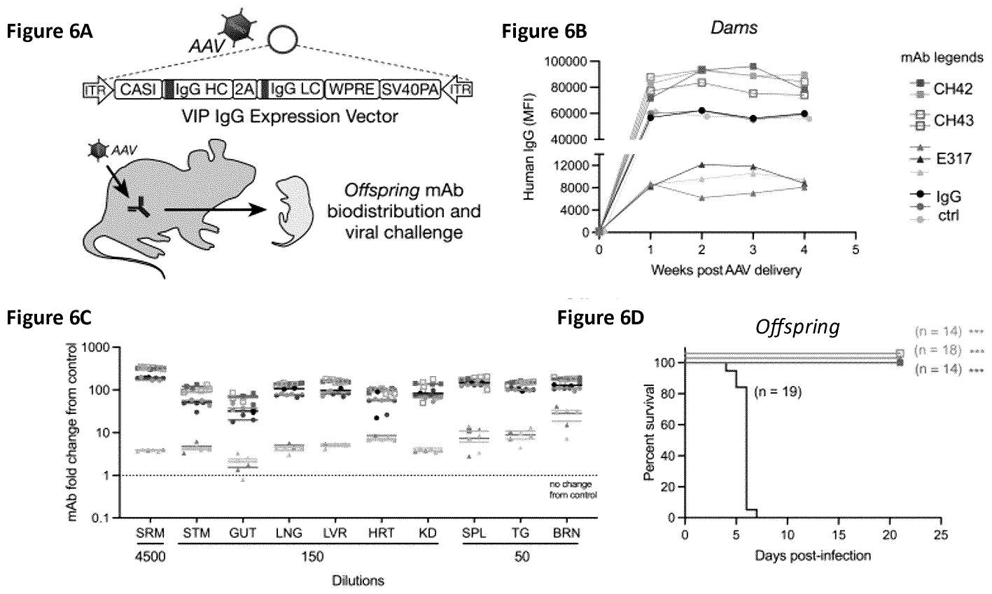

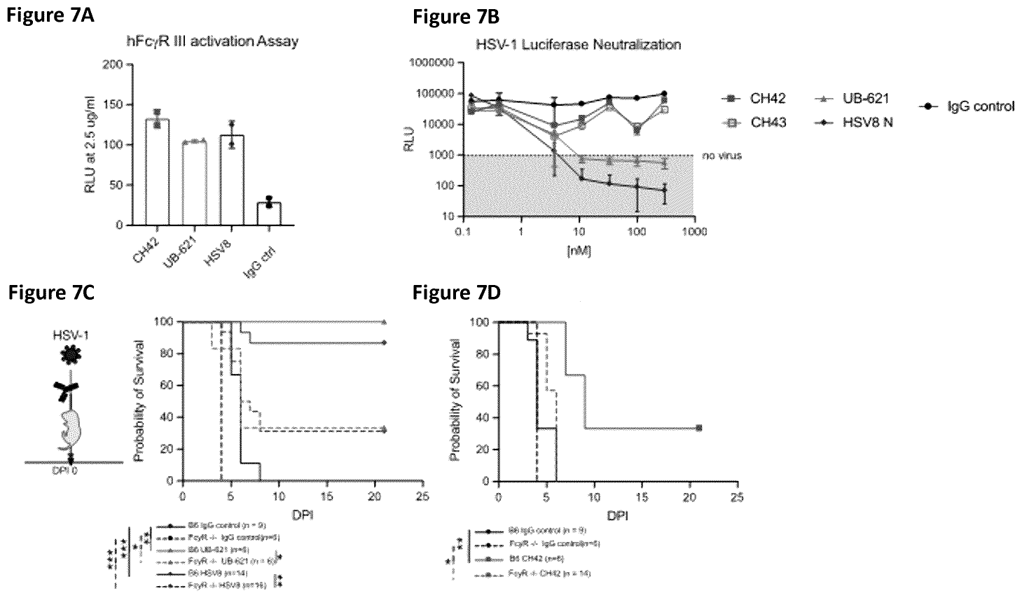

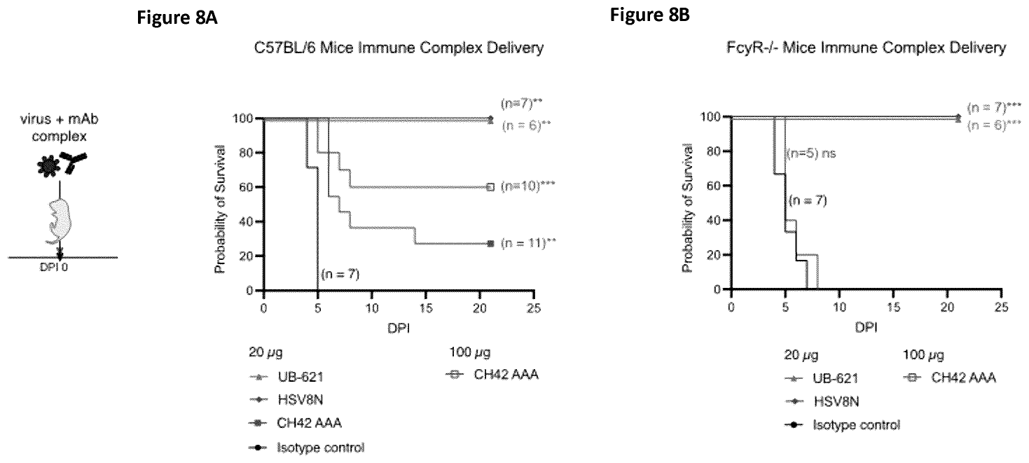

HSV-specific mAbs delivered via vectored immunoprophylaxis (VIP) provide trans-generational protection from nHSV mortality. Having shown that administration of mAbs to dams protects their pups from nHSV mortality, we sought to investigate vectored antibody delivery using AAV. Female mice received a single intramuscular injection of AAV vectors each encoding a human mAb (FIG. 6A). Serum was obtained over a four-week period to confirm mAb expression (FIG. 6B) via immunoassay. All transduced dams expressed huIgG in the serum at different levels, over a period of approximately 6 months and 3 pregnancies. All progeny of VIP-transduced dams had detectable levels of mAbs in serum (FIG. 6C). Furthermore, mAb was effectively transferred throughout visceral organs and the nervous system (FIG. 6C). The biodistribution of mAbs was similar to that observed in neonatal mice directly injected i.p. Offspring of E317 VIP-transduced dams had the lowest levels of mAb expression in all organs, consistent with its low expression in the dams.

Finally, to assess whether the transferred mAbs were sufficient to protect pups from lethal viral challenge, progeny of VIP-transduced dams were challenged with HSV-1 two days postpartum, and monitored until weaning at three weeks of age. Progeny of HSV mAb VIP-transduced dams were completely protected from mortality, while progeny of control IgG VIP-transduced dams rapidly succumbed to infection (FIG. 6D). These findings demonstrate that antibodies produced in dams via VIP and subsequently transferred to pups were protective (p<0.001). Moreover, although pups from E317 VIP-transduced dams received considerably less mAb, they were equally protected from disease as pups receiving 10-50-fold higher levels of mAbs from their mothers. Together, these findings further underscore the promise of diverse mAb delivery strategies in protection against nHSV-induced morbidity and mortality.

### Example 2: Antibody Fc-Mediated Functions are Critical for Neonatal Herpes Simplex Virus Infection Survival

Neonatal viral infections account for an estimated 6.5% of newborn deaths, some of which can be prevented by the passive immunity afforded to the neonate via maternal antibodies (Ab) in the first 6 months of life. Primary Herpes Simplex Virus (HSV) infections in late pregnancy account for the majority (>80% risk) of neonatal HSV infections, suggesting that maternal Abs greatly dimmish the risk of neonatal infection (<1%). Neonatal HSV infections have the highest fatality rate among neonatal infections, therefore, understanding which Ab-dependent immune functions protect from severe illness, can direct us towards better maternal vaccination strategies and personalized therapeutic development to reduce neonatal mortality.

It remains unclear if the neonatal immune response supports robust Fc-dependent effector functions. Therefore, we tested the hypothesis that neutralization and Fc-mediated effector functions worked in concert to protect neonates from mortality associated with neonatal HSV infection. HSV-specific monoclonal antibodies (mAbs) with varying neutralization potencies were used to better understand Ab-mediated functions in a mouse model of neonatal infection. Well characterized mutations in the Fc region of mAbs, combined with mice lacking Fcγ receptors (FcγRs) allowed for evaluation of different Fc mediated functions. Neonatal mice received mAbs with and without modifications, were challenged with HSV-1, and were assessed for survival. In addition, neonatal mice were challenged with bioluminescent reporter HSV-1 to determine the temporal effects of mAb treatment.

HSV mAbs protect neonatal mice from mortality and decrease viral replication. Furthermore, mice have increased susceptibility to infection when FcγRs cannot be engaged. Modified Ab-Fc that cannot activate FcγRs or complement results in increased mortality. In parallel, FcγR deficient mice had increased mortality to infections, suggesting that engagement of antibody Fc through FcγR and complement is an important protective mechanism in neonates. Therefore, prophylactic and therapeutic interventions should consider maximal engagement of these protective Ab-dependent mechanisms.

These studies provide evidence that Fc mutations impact vFcR binding, neutralization potency, and in vivo efficacy. gE/gI mutant (and revertant) HSV-1 strain NS-gE264 are sufficiently infective and pathogenic to support in vitro and in vivo assessment.

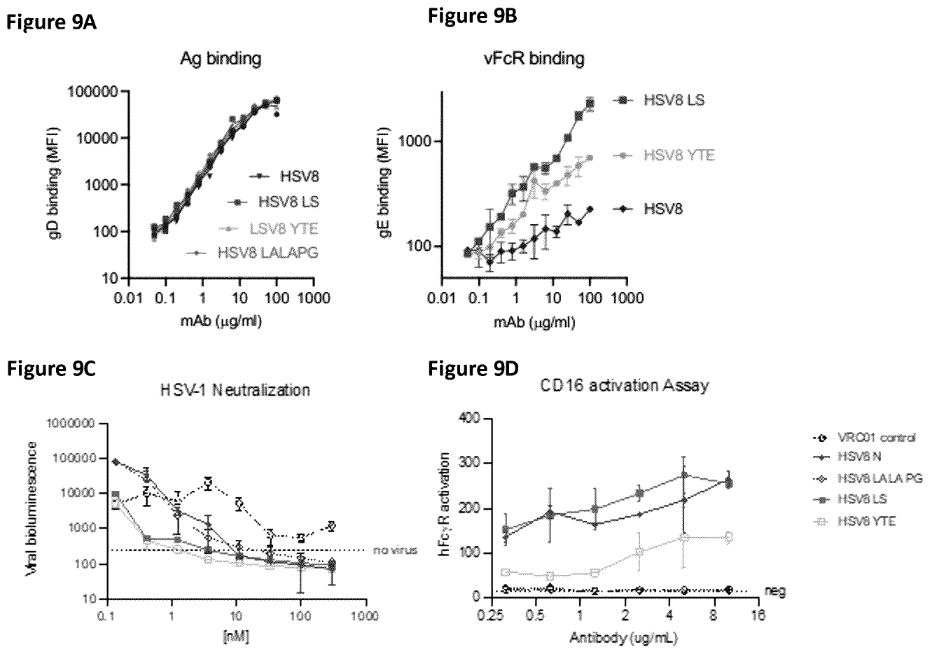

As shown in FIG. 9A, HSV8 and Fc mutants-HSV8 LS, HSV8 YTE, and HSV8 LALAPG—displayed equivalent binding to gD in a bead-based multiplex assay. Fc mutations LS and YTE increased binding to recombinant gE, a component of the vFcR complex over a wide titration range in a bead-based multiplex assay. These mAb Fc variants displayed different neutralizing potency and effector activation profiles which may be influenced by the vFcR.

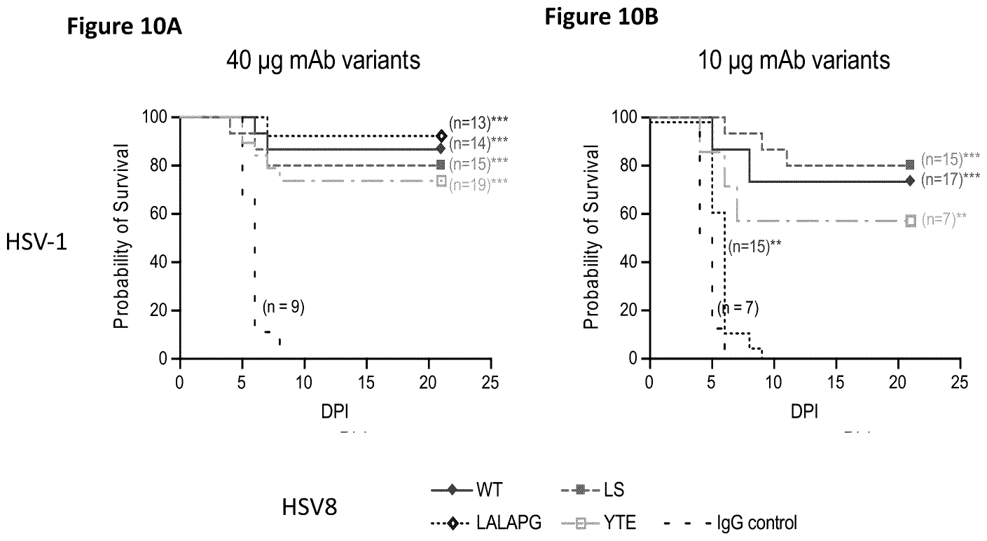

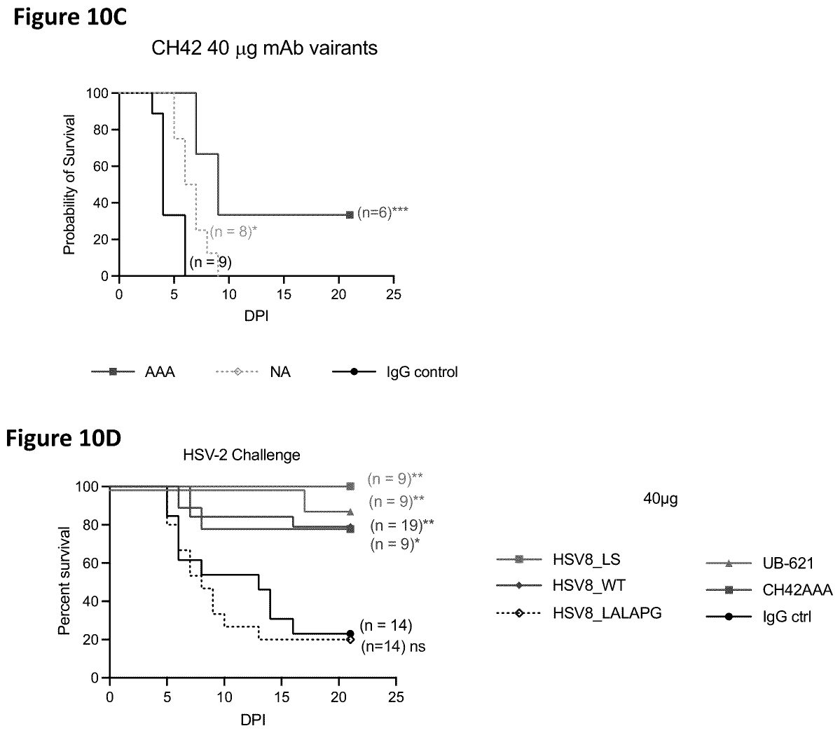

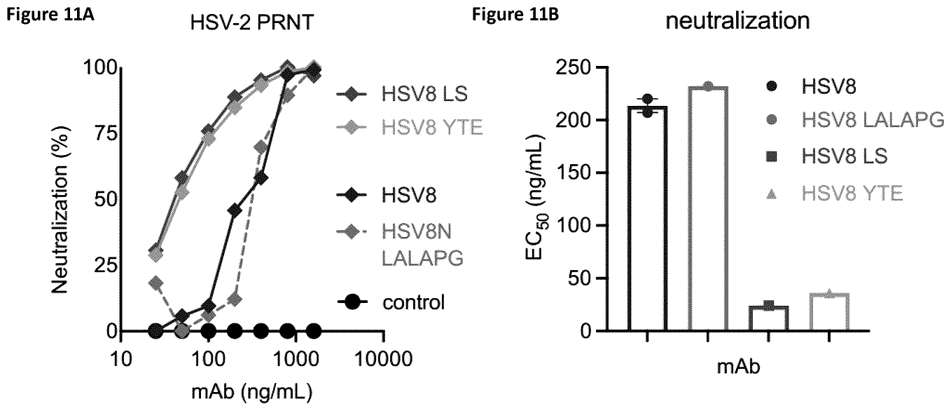

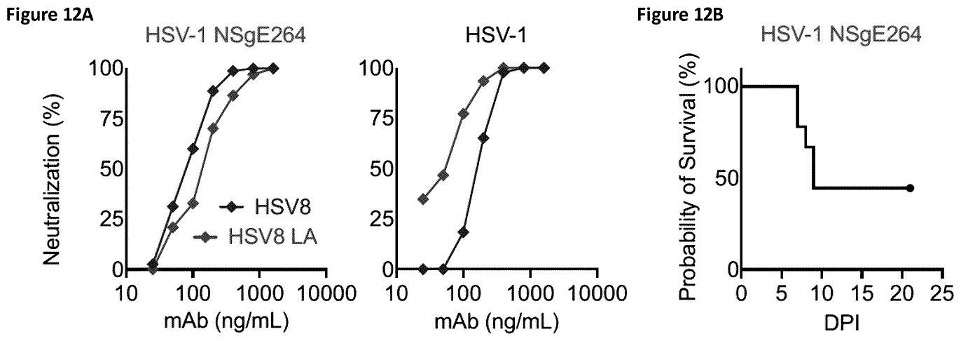

As shown in FIG. 10, survival after lethal HSV-1 challenge was roughly equivalent for wild-type antibody and Fc mutants at the higher dose level. At the lower dose level, survival differences emerged after lethal HSV-1 challenge for Fc mutants. Thus, at lower mAb concentrations Fc-mediated functions may become increasingly important.

These studies demonstrate that mAb neutralization potency and in vivo efficacy is enhanced by YTE, LS, and LA Fc domain mutations.

### Example 3: Antibodies with Fc Mutations that Enhance Binding to the Viral Fc Receptor (gE) Exhibit Improved In Vitro and In Vivo Activity

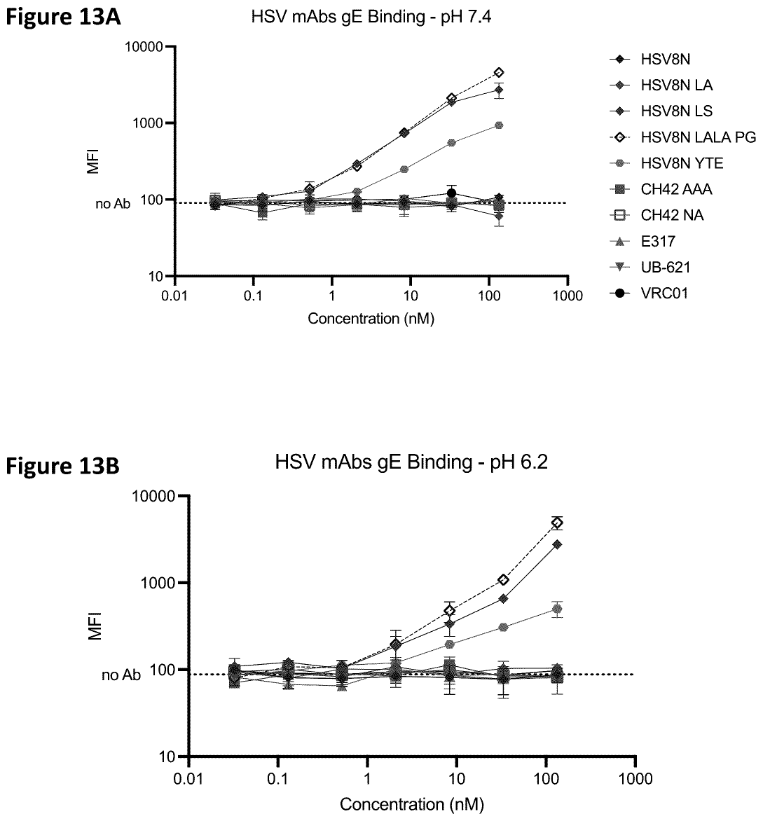

HSV-1 gE binding to mAbs was evaluated in a multiplexed bead-based assay at two distinct pH conditions 7.4 and 6.2. Briefly, gE-coupled beads were incubated with monoclonal antibodies overnight before being detected with a mouse anti-IgG Hinge antibody. Binding was measured via a flexmap 3D instrument and reported as mean fluorescent intensity (MFI). Data is shown in FIG. 13A-13B.

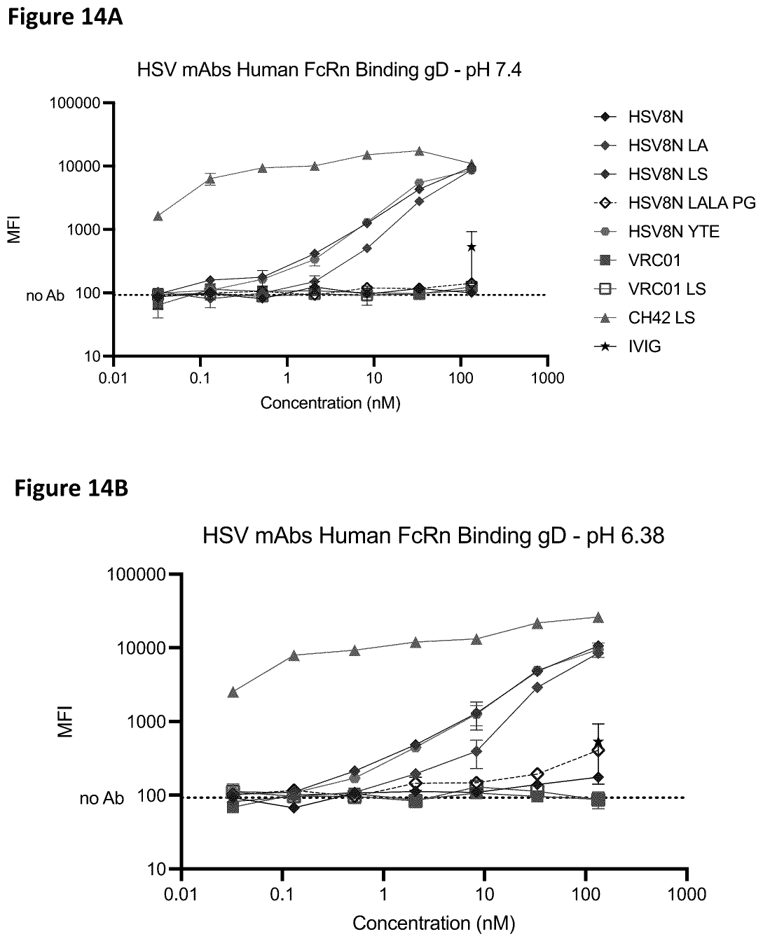

Human FcRn binding to monoclonal antibodies was measured via a multiplex bead-based assay. Briefly, serially diluted mAbs were incubated with beads coupled with HSV-2 gD. Immune complexes were washed before being incubated with human FcRn tetramerized with streptavidin-PE. Binding was measured via a Flexmap 3D instrument and values were reported as mean fluorescent intensity (MFI). Data is shown in FIG. 14A-14B.

HSV-1 strain NS is a low-passage clinical isolate. See Friedman, H. M., E. J. Macarak, R. R. MacGregor, J. Wolfe, and N. A. Kefalides. 1981. Virus infection of endothelial cells. J. Infect. Dis. 143:266-273.

HSV-1 NS-gE264 (gE Mutant) contains a 4 amino acid insertion after gE residue 264, based on the sequence of HSV-1 strain 17 (after gE amino acid 266 in HSV-1 strain NS, which has two additional amino acids at gE positions 186 and 187 compared to strain 17). HSV-1 NS-gE264 maintains gE activity in vivo but eliminates binding to human IgG Fc. See Lubinski, J. M., Lazear, H. M., Awasthi, S., Wang, F., Friedman, H. M., 2011. The Herpes Simplex Virus 1 IgG Fc Receptor Blocks Antibody-Mediated Complement Activation and Antibody-Dependent Cellular Cytotoxicity In Vivo. Journal of Virology 85, 3239-3249.

HSV-1 rNS-gE264 (gE Rescue) is a rescue virus generated by co-transfecting gE mutant with plasmid encoding for the entire gE protein and screening for loss of insertion. Id.

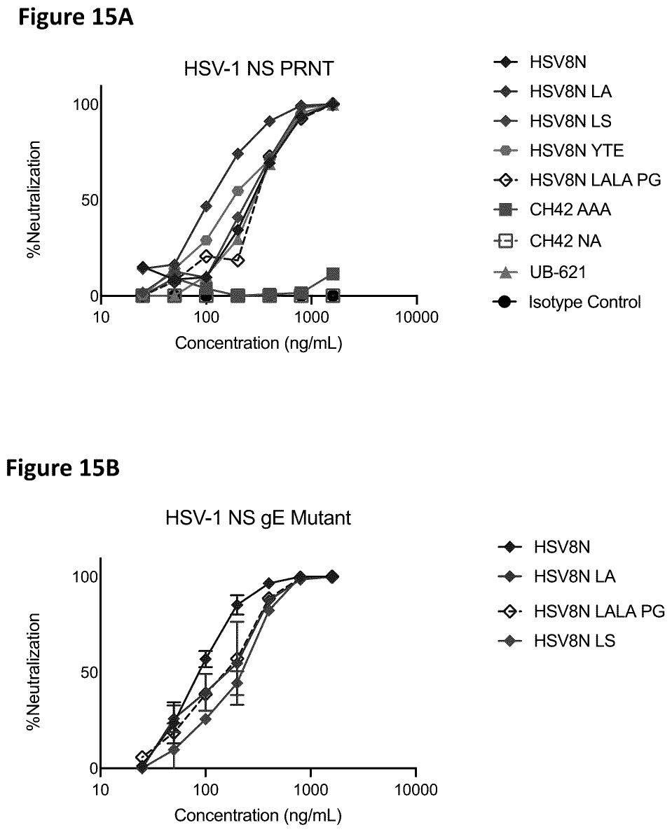

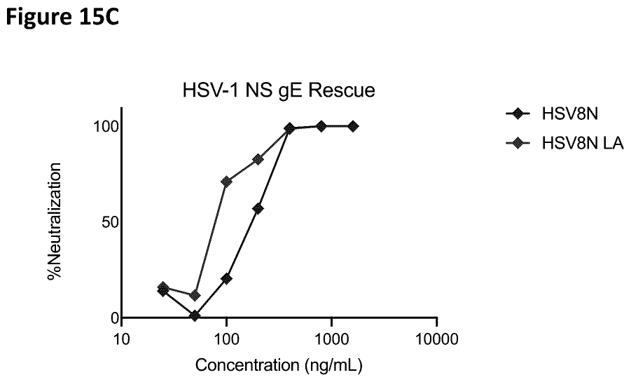

Antibody neutralization potency was measured via Plaque Reduction for three HSV variants, WT NS, NS-gE264 (gE mutant), and rNS-gE264 (gE Rescue). Briefly, serially diluted antibody was incubated with 100 μL of 1e3 PFU/mL HSV for one hour before being added to Vero cell monolayers in 6 well plates. Virus was allowed to adsorb for 1 hour with agitation before 2 mL of a methylcellulose overlay was added. Plaques were allowed to form for 72-96 hours before cells were fixed, stained, and plaques were counted. Data is shown in FIG. 15A-15C.

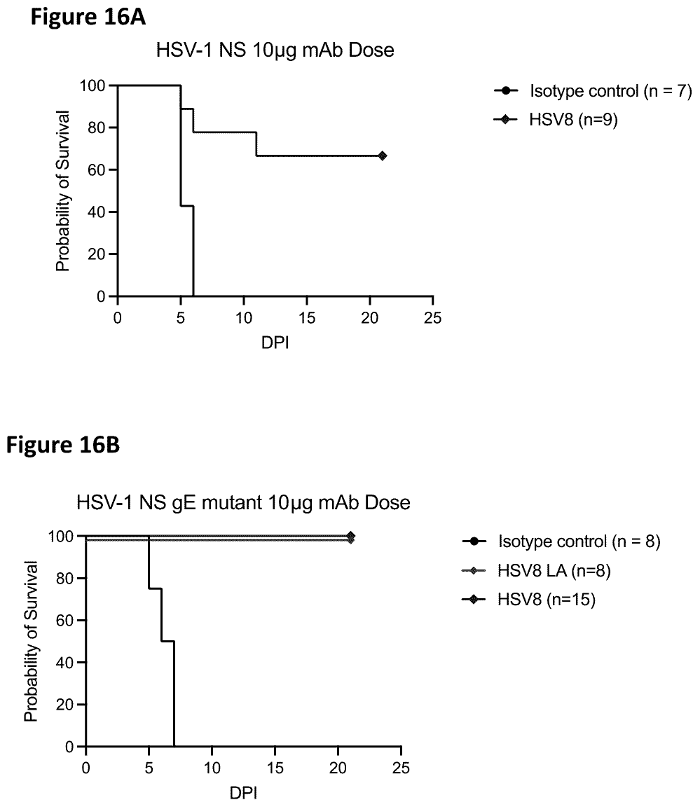

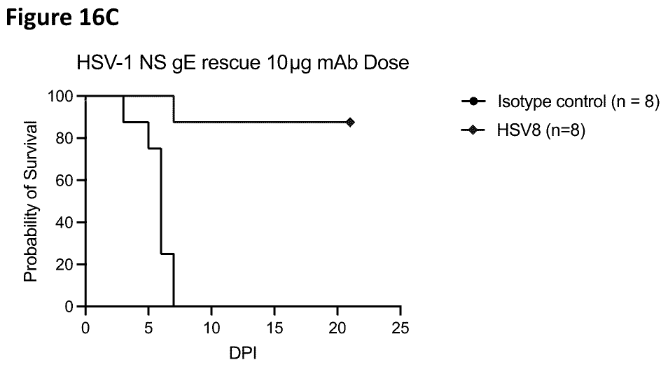

Ability for HSV gE to bind to IgG Fc affects mAb efficacy in vivo. 2-day old wild type C57BL/6J mice were treated with 10 ug of HSV8, HSV8 LA, or an isotype control (VRC01) intraperitoneally prior to a 1e4 PFU viral challenge intranasally. Pups were monitored for survival for 21 days. Data is shown in FIG. 16A-16C.

1. A method for preventing or ameliorating the effects of a neonatal herpes simplex virus (HSV) infection comprising: (a) administering to a maternal subject that is pregnant or likely to become pregnant an anti-HSV antibody or (b) administering to a neonate infected with a HSV, at risk for being infected with a HSV, or that has been exposed to a HSV an anti-HSV antibody, wherein the anti-HSV antibody comprises an Fc region having at least one modification or mutation that confers (a) enhanced effector function and/or (b) improved viral Fc receptor (vFcR) and/or glycoprotein E (gE) binding properties relative to a wild-type Fc region.

2. The method of claim 1, wherein the modification or mutation comprises an amino acid mutation in the Fc region relative to wild-type IgG1 Fc region, wherein the wild-type IgG1 Fc region optionally comprises the amino acid sequence of SEQ ID NO: 28.

3. The method of claim 2, wherein the anti-HSV antibody comprises an Fc region having the LS (i.e., M428L/N434S) mutation; an Fc region having the LA (i.e., M428L/N434A) mutation; or an Fc region having the YTE (i.e., M252Y/S254T/T256E) mutation.

4. The method of claim 1, wherein the anti-HSV antibody comprises an afucosylated Fc region.

5. The method of claim 1, wherein the enhanced effector function comprises enhanced antibody dependent cellular cytotoxicity (ADCC), enhanced antibody dependent cellular phagocytosis (ADCP), and/or enhanced complement-dependent cytotoxicity (CDC).

6. The method of claim 1, wherein the anti-HSV antibody has the binding specificity of mAb 5188, E317, or HSV8.

7. The method of claim 1, wherein the anti-HSV antibody comprises
a heavy chain variable region (VH) having
a V_{H} CDR1 having an amino acid sequence of SEQ ID NO: 1,
a V_{H} CDR2 having an amino acid sequence of SEQ ID NO: 2, and
a V_{H} CDR3 having an amino acid sequence of SEQ ID NO: 3; and
a light chain variable region (VL) having
a V_{L} CDR1 having an amino acid sequence of SEQ ID NO: 4,
a V_{L} CDR2 having an amino acid sequence of SEQ ID NO: 5, and
a V_{L} CDR3 having an amino acid sequence of SEQ ID NO: 6.

8. The method of claim 1, wherein the anti-HSV antibody comprises a heavy chain variable region (VH) having an amino acid sequence of SEQ ID NO: 7 and a light chain variable region (VL) having an amino acid sequence of SEQ ID NO: 8.

9. The method of claim 1, wherein the anti-HSV antibody comprises
a heavy chain variable region (VH) having
a V_{H} CDR1 having an amino acid sequence of SEQ ID NO: 10,
a V_{H} CDR2 having an amino acid sequence of SEQ ID NO: 11, and
a V_{H} CDR3 having an amino acid sequence of SEQ ID NO: 12; and
a light chain variable region (VL) having
a V_{L} CDR1 having an amino acid sequence of SEQ ID NO: 13,
a V_{L} CDR2 having an amino acid sequence of SEQ ID NO: 14, and
a V_{L} CDR3 having an amino acid sequence of SEQ ID NO: 15.

10. The method of claim 1, wherein the anti-HSV antibody comprises a heavy chain variable region (VH) having an amino acid sequence of SEQ ID NO: 16 and a light chain variable region (VL) having an amino acid sequence of SEQ ID NO: 17.

11. The method of claim 1, wherein the anti-HSV antibody comprises
a heavy chain variable region (VH) having
a V_{H} CDR1 having an amino acid sequence of SEQ ID NO: 20,
a V_{H} CDR2 having an amino acid sequence of SEQ ID NO: 21, and
a V_{H} CDR3 having an amino acid sequence of SEQ ID NO: 22; and
a light chain variable region (VL) having
a V_{L} CDR1 having an amino acid sequence of SEQ ID NO: 23,
a V_{L} CDR2 having an amino acid sequence of SEQ ID NO: 24, and
a V_{L} CDR3 having an amino acid sequence of SEQ ID NO: 25.

12. The method of claim 1, wherein the anti-HSV antibody comprises a heavy chain variable region (VH) having an amino acid sequence of SEQ ID NO: 26 and a light chain variable region (VL) having an amino acid sequence of SEQ ID NO: 27.

13. The method of claim 1, wherein the anti-HSV antibody comprises the heavy chain CDRs and the light chain CDRs of HSV8.

14. The method of claim 1, wherein the anti-HSV antibody comprises the heavy chain variable region (VH) and light chain variable region (VL) of HSV8.

15. The method of claim 1, wherein the maternal subject is HSV seronegative.

16. The method of claim 1, wherein the maternal subject is suspected of having a primary HSV infection.

17. The method of claim 1, wherein the maternal subject is pregnant.

18. The method of claim 1, wherein the anti-HSV antibody is administered to the maternal subject prior to parturition.

19. An anti-herpes simplex virus (HSV) antibody comprising an Fc region having at least one modification or mutation that confers (a) enhanced effector function and/or (b) altered (e.g., improved) viral Fc receptor (vFcR) and/or glycoprotein E (gE) binding properties relative to a wild-type Fc region, wherein the anti-HSV antibody is optionally for use in a method for preventing or ameliorating the effects of a neonatal HSV infection.

20. The anti-HSV antibody of claim 19, wherein the modification or mutation confers altered (e.g., improved) viral Fc receptor (vFcR) and/or glycoprotein E (gE) binding properties relative to a wild-type Fc region and wherein the modification or mutation comprises (a) afucosylation and/or (b) an amino acid mutation in the Fc region relative to wild-type IgG1 Fc region, wherein the wild-type IgG1 Fc region optionally comprises the amino acid sequence of SEQ ID NO: 28 and wherein the amino acid mutation is optionally selected from the group consisting of LS (i.e., M428L/N434S); LA (i.e., M428L/N434A); and YTE (i.e., M252Y/S254T/T256E).

21. The anti-HSV antibody of claim 19, wherein the anti-HSV antibody comprises
a heavy chain variable region (VH) having
a V_{H} CDR1 having an amino acid sequence of SEQ ID NO: 20,
a V_{H} CDR2 having an amino acid sequence of SEQ ID NO: 21, and
a V_{H} CDR3 having an amino acid sequence of SEQ ID NO: 22; and
a light chain variable region (VL) having
a V_{L} CDR1 having an amino acid sequence of SEQ ID NO: 23,
a V_{L} CDR2 having an amino acid sequence of SEQ ID NO: 24, and
a V_{L} CDR3 having an amino acid sequence of SEQ ID NO: 25; and
wherein the modification or mutation comprises (a) afucosylation and/or (b) an amino acid mutation in the Fc region relative to wild-type IgG1 Fc region, wherein the wild-type IgG1 Fc region optionally comprises the amino acid sequence of SEQ ID NO: 28 and wherein the amino acid mutation is optionally selected from the group consisting of LS (i.e., M428L/N434S); LA (i.e., M428L/N434A); and YTE (i.e., M252Y/S254T/T256E).

22. The anti-HSV antibody of claim 21, wherein the modification or mutation comprises afucosylation, the LS amino acid mutation, or the LA amino acid mutation.

23. (canceled)

24. (canceled)

25. The anti-HSV antibody of claim 21, wherein the anti-HSV antibody comprises a heavy chain variable region (VH) having an amino acid sequence of SEQ ID NO: 26 and a light chain variable region (VL) having an amino acid sequence of SEQ ID NO: 27.
